(19) United States
(12) Patent Application Publication
(10) Pub. No.: US 2019/0276793 A1
(43) Pub. Date: Sep. 12, 2019

(54) Methods and Apparatus for Regulation of Gene Expression Across a Large-Scale Solid Structure

(71) Applicant: Massachusetts Institute of Technology, Cambridge, MA (US)

(72) Inventors: Christoph Bader, Cambridge, MA (US); Rachel Smith, Cambridge, MA (US); Sunanda Sharma, Shrewsbury, MA (US); Dominik Kolb, Kisslegg (DE); Neri Oxman, Cambridge, MA (US)

(21) Appl. No.: 16/295,502

(22) Filed: Mar. 7, 2019

Related U.S. Application Data
(60) Provisional application No. 62/639,528, filed on Mar. 7, 2018.

(51) Int. Cl. C12N 1/20; C12N 1/38

(52) U.S. Cl. CPC C12N 1/20; C12N 1/38; C12N 2533/30; C12N 2510/00; C12N 2535/10

(57) ABSTRACT

A 3D printer may precisely control deposition of diffusible chemical signals in different spatial regions of a solid polymer structure, in such a way that the concentration and spatial distribution of each diffusible chemical signal in each spatial region of the structure is independently controlled. A hydrogel containing genetically engineered, living organisms may be applied to a surface of the solid polymer structure. The living organisms may be single-celled organisms, such as bacteria. The diffusible chemical signals may diffuse out of the solid polymer structure and into the hydrogel, and may control gene expression of genetically engineered cells in different spatial locations in the hydrogel. Thus, gene expression of genetically-engineered cells in a hydrogel may be controlled on a region-by-region basis, by precisely controlling the position and concentration of diffusible chemical signals that are initially embedded in a solid structure adjacent to the hydrogel.

### RELATED APPLICATIONS

This application claims the benefit of U.S. Provisional Application No. 62/639,528 filed Mar. 7, 2018 (the “Provisional”).

### FIELD OF TECHNOLOGY

The present invention relates generally to regulation of gene expression.

### SUMMARY

In illustrative implementations, a HLM (hybrid living material) includes: (a) a solid polymer structure; (b) diffusible chemical signals that are embedded in the solid structure; (c) a thin hydrogel coating that is in physical contact with a smooth surface of the solid structure; and (d) living organisms (e.g., single-celled organisms) that are surrounded by the hydrogel.

The hydrogel may comprise a thin aqueous gel that conforms to, touches, and coats the solid polymer structure. The living organisms may be dispersed throughout the hydrogel and may be inside the hydrogel. The hydrogel may contain nutrients and growth media for the organisms. The hydrogel may provide transport for diffusible chemical signals and a hospitable environment in which the organisms may grow.

The living organisms may be genetically engineered in such a way that gene expression of the organisms is controlled by the diffusible chemical signals.

The HLM may regulate gene expression of the living organisms in such a way as to control production of proteins, RNA, or enzymatic reactions in different spatial regions of the structure surface, in relation to diffusible chemical concentration.

In some cases, the polymer structure (and its hydrogel coating) are large. For instance, in some cases, polymer structure (and its hydrogel coating) each have a maximum dimension that is greater than or equal to 1 millimeter and less than or equal to 1 meter. Thus, in some cases, the HLM regulates gene expression in a hydrogel that coats a large surface of the solid polymer structure.

A 3D printer may fabricate the solid polymer structure. The 3D printer may, in accordance with digital instructions, precisely control the amount and type of materials that are deposited to form different spatial regions of the solid structure. For instance, the 3D printer may, when fabricating the structure, control where and in what concentration each chemical signal is deposited. The 3D printer may deposit the chemical signals in continuous (or discrete, step-wise) gradients in such a way that each chemical signal has a different distribution of concentrations throughout the structure. Also, the 3D printer may control the material properties (e.g. absorbency, diffusivity, hardness, rigidity, tensile strength, compressive strength, transparency, color) of the polymer structure itself, in such a way that these material properties vary as a function of spatial position in the structure. For instance, the 3D printer may deposit materials in such a way that the polymer structure itself has a continuous (or discrete, step-wise) gradient of material properties. Also, for instance, the 3D printer may deposit materials in such a way as to cause: (a) some regions of the polymer structure to be hard and rigid and other regions of the polymer structure to be soft and flexible; and (b) some regions of the polymer structure to have a higher diffusivity (and thus to facilitate more rapid diffusion of embedded chemical signals) than other regions of the polymer structure. A non-limiting example of “diffusivity” is the degree to which the polymer structure permits diffusion of the chemical signals inside the polymer structure.

The 3D printer may fabricate the polymer structure by depositing materials in such a way that each of a set of material properties (and each chemical signal) has an independent spatial distribution in the polymer structure. For instance, the spatial distribution of the absorbency or diffusivity of the polymer structure may vary independently from the spatial distribution of each embedded chemical signal.

In some implementations, the solid structure comprises one or more photopolymers (e.g., photo-curable acrylic or acrylate polymers) The 3D printer may control material properties (e.g., hardness, rigidity, diffusivity, absorbency, translucency, color) of the fabricated object by depositing different combinations of materials in different relative proportions. For instance, the 3D printer may produce different material properties by depositing different mixtures, composites or blends of acrylate polymers (or of additives and acrylate polymers).

When exposed to water, the cured polymer structure may take up (absorb) a portion of the water. For instance, when touching the hydrogel coating, the polymer structure may absorb a portion of the water that is in the hydrogel. The addition of water to the cured polymer structure may in turn enable the embedded chemical signals to diffuse. For instance, the diffusible chemical signals, which were initially embedded in the polymer structure, may diffuse through and out of the polymer structure. The system's diffusivity (or the degree to which the polymer structure facilitates the diffusion of the chemical signals) may vary as a function of spatial position in the polymer structure.

In illustrative implementations, the 3D printer precisely controls gene expression of the genetically engineered organisms (e.g., single-celled organisms) that are spatially dispersed in the hydrogel coating at different spatial regions relative to the solid structure surface. The 3D printer may do so by precisely controlling deposition of diffusible chemical signals in different spatial regions of the solid structure, in such a way that: (a) the concentration of each diffusible chemical signal in each spatial region of the printed solid structure is independently controlled, (b) the concentration of diffusible enabling physical properties in each spatial region of the printed solid structure is independently controlled, and (c) the distribution of each chemical signal that is embedded in the structure is different than that of each other chemical signal that is embedded in the structure

Thus, in illustrative implementations, gene expression of genetically-engineered cells in a hydrogel coating is controlled on a region-by-region basis, by precisely controlling the position and concentration of diffusible chemical signals that are embedded in a solid polymer structure adjacent to the hydrogel.

In some cases, the immobilization of many gene-encoded sensors across the surface of the 3D-printed polymer structure helps create a multi-sensor construct (e.g., a multicellular sensing construct) across the surface of the 3D-printed polymer structure. This multi-sensor construct may respond to specific signals which diffuse from the polymer structure, and thus produce patterned outputs that alter the polymer surface selectively and in a programmed manner.

In some cases, the responses of each of the spatially distributed, immobilized biological agents, based on their individual exposure profile to the dynamic inducer diffusion fields, result in a collective pattern.

In some cases, each chemical signal functions as an interfacing signal to relate digitally-controllable material property commands from the abiotic polymer structure to inducer and stimuli responsive, genetically-encoded biotic “sensors” distributed across the hydrogel-coated object surface.

In some cases, genetically engineered cells are substantially immobilized in the hydrogel portion of the HLM. This immobilization, in turns, facilitates the spatial control of gene expression, since the immobilization substantially fixes the position of the engineered cells relative to the solid polymer object from which chemical signals are diffusing in a spatial pattern.

In some cases, the solid polymer structure does not have any pores or internal cavities (or has only pores or cavities that are much smaller than the single-celled organisms in the hydrogel). In some cases: (a) the 3D printer deposits liquid resin droplets that spread before they are cured by UV light; and (b) this spreading of the liquid droplets causes the solid polymer object to have a smooth surface and to not have any macro-scale pores or internal cavities.

In some cases, the solid polymer structure is not biodegradable. In some cases, the hardness and lack of porosity of the solid polymer structure is permanent (e.g., persists for years).

The hybrid living material (HLM) of the present invention is quite different than conventional tissue scaffolds. Among other things, in many conventional tissue scaffolds: (a) the scaffold is a soft hydrogel that lacks structural strength (e.g., cannot support its weight or additional external load, lacks rigidity, or can be easily torn or ripped apart); (b) the scaffold has pores and cavities that penetrate through its surface, thereby allowing cells to infiltrate and proliferate within the scaffold; and (c) the scaffold is designed to degrade over time and to be replaced by the proliferating cells (and by extracellular material created by the cells). In contrast, in many implementations of the present invention: (a) all or part of the solid polymer structure is hard and structurally strong; (b) the solid structure has a smooth surface; (c) the solid structure does not have any pores or cavities that are larger than smallest living cell in the hydrogel; (d) cells do not enter and proliferate inside the solid structure; (e) the solid structure does not degrade; and (f) the solid structure permanently (e.g., for years) retains its shape, hardness, smoothness and lack of large-scale pores and cavities.

In some implementations of this invention, genetically-engineered living organism act as signal filters, distributed as nodes of a multi-sensor system across a physical 3D surface. These organisms may be genetically-engineered to be stimuli-responsive to specific single or multiple input commands. These organisms may process input chemical signals. For instance, these organisms may amplify a signal, invert a signal, create temporal functions (e.g. like an oscillator), count signals (e.g., like a state machine), or create conditionals based on neighboring agents' signals (e.g. quorum). Furthermore, any input condition may be placed in control of any output function, such as the creation of a protein or RNA, the synthesis metabolization of a compound, or the modulation of enzymatic activity.

This invention has many practical applications. For instance, in some implementations, this invention controls gene expression in an HLM in such a way that the HLM produces protein, RNA, enzymes, or metabolically synthesized compounds. In some cases, the structural strength of the solid polymer component of the HLM enables the HLM to have a large size and to output industrial quantities of these products.

The Summary and Abstract sections and the title of this document: (a) do not limit this invention; (b) are intended only to give a general introduction to some illustrative implementations of this invention; (c) do not describe all of the details of this invention; and (d) merely describe non-limiting examples of this invention. This invention may be implemented in many other ways. Likewise, the Field of Technology section is not limiting; instead it identifies, in a general, non-exclusive manner, a field of technology to which some implementations of this invention generally relate.

### BRIEF DESCRIPTION OF THE DRAWINGS

FIG. 1 is a conceptual diagram of different blends of materials.

FIG. 2 shows a 3D printer that is printing a solid, polymer structure, approximately half a meter in length.

FIG. 3 shows a solid, polymer structure and the position of different continuous material blends within the structure.

FIG. 4 is an exploded view of a solid, polymer structure and a hydrogel that has been applied to the surface of the structure.

FIG. 5 shows diffusion of embedded chemicals.

FIG. 6 shows a spatial distribution of gene expression on the object, induced by diffused chemicals.

FIG. 7 shows a use case, in which chemicals embedded at programmed locations in a back brace interact with an inducible biological system to deliver spatiotemporally patterned or regulated therapeutic drug production to the surface of a person's back to treat a medical condition.

FIG. 8 is a flowchart of a method of controlling gene expression by diffusion of chemicals that are embedded in a polymer structure.

The above Figures are not necessarily drawn to scale. The above Figures show illustrative implementations of this invention, or provide information that relates to those implementations. The examples shown in the above Figures do not limit this invention. This invention may be implemented in many other ways.

### DETAILED DESCRIPTION

### General

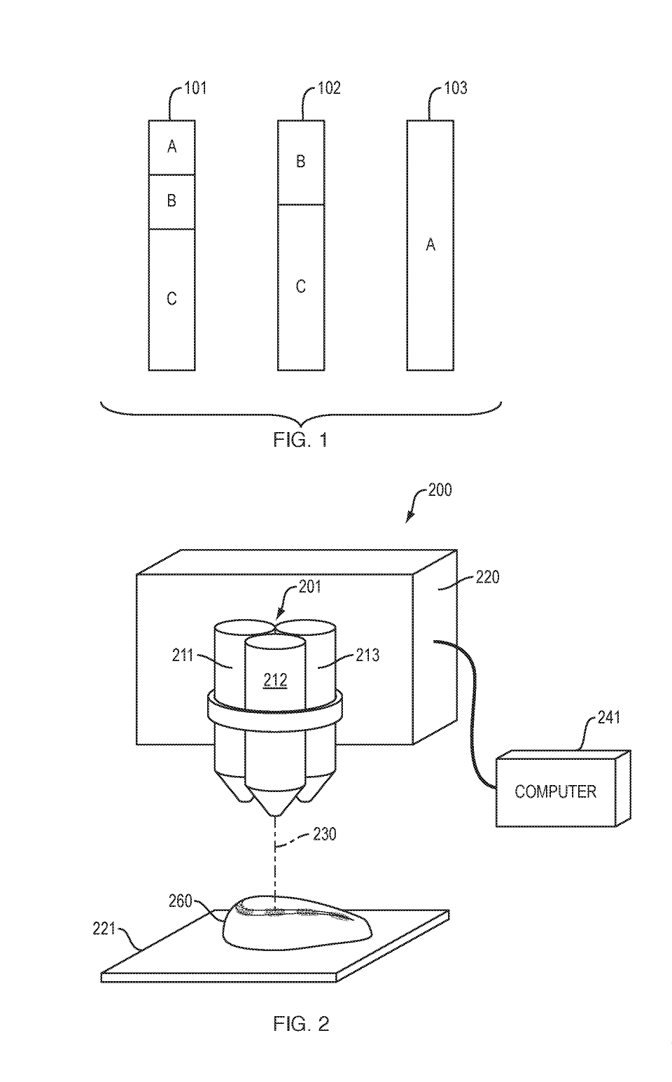

In illustrative implementations of this invention, a 3D printer prints different materials or different mixtures of materials, each from different nozzle(s) or jet(s). FIG. 1 is a conceptual diagram of different blends of materials that may each be printed from separate nozzle(s) or jet(s) of a 3D printer (e.g., an inkjet 3D printer). In the example shown in FIG. 1: (a) composite 101 comprises a mixture of material A, material B and material C; (b) composite 102 comprises a mixture of material B and material C; and (c) material 103 consists of only material A. For instance, one or more of materials A, B and C may comprise a photopolymer.

In FIG. 1, the relative sizes of the areas occupied by the ingredients signify the relative proportions of the ingredients (and are not intended to indicate whether the materials are each spatially separate from each other). In FIG. 1, each respective mixture (101, 102) may comprise: (a) an evenly mixed blend of multiple materials, (b) a continuous spatial gradient from one material to another, or (c) a discrete (step-wise) or dithered transition from one material to another. FIG. 1 shows a non-limiting example. Other combinations of materials, in other relative proportions, may be employed instead.

In illustrative implementations, the 3D printer fabricates a solid, polymer structure. This structure may be, at least in some regions of the structure, hard and rigid. This structure may have a smooth external surface and may lack macro-scale pores and cavities.

FIG. 2 shows a 3D printer 200 that is printing a solid, polymer structure 260. The 3D printer includes a printhead 201. The printhead 201 comprises multiple print units (e.g., 211, 212, 213). Each print unit (e.g., 211, 212, 213) includes: (a) a chamber for storing, mixing, heating or cooling material that will be deposited by the print unit; and (b) nozzle(s) or jet(s) to deposit the material (e.g., by extruding, ejecting or jetting the material). Each print unit may deposit a different material (e.g., a different compound or a different mixture of compounds). The 3D printer includes a build platform 221. Object 260 rests on build platform 221 while being fabricated. The 3D printer includes one or more actuators 220 (e.g., electric motors and structures for transmitting motion or force) that actuate motion of printhead 201 while printhead 201 is depositing material to fabricate object 260. The material exiting each nozzle may comprise a stream, jet, droplet or filament 230 of material (e.g., photopolymers). A computer 241 may control the 3D printer 200, including: (a) controlling actuators 220 and thereby controlling movement and trajectory of printhead 201; and (b) controlling printhead 201 itself and thereby controlling when, what location, and what materials are ejected from printhead 201.

In some implementations, the print instructions (e.g., droplet deposition instructions) for the 3D printer are generated based on a digital model. This digital model may specify surface geometry and internal material distributions of a solid polymer structure (which will form the structural framework of the HLM). The digital model may specify the placement and concentration of chemical signals and chemical-retaining/water-absorbent matrix throughout the solid polymer structure.

The 3D printer may fabricate a solid polymer structure by multimaterial 3D printing. A computer may generate droplet deposition instructions of chemical signaling photopolymer resins and water-absorbent resins, based on geometry and concentrations of chemical signals that are specified in the digital model.

The chemical signaling resins may be loaded into the 3D printer and then deposited in accordance with the droplet deposition instructions, to form a solid, polymer structure.

Resins may be mixed or combined before or during the 3D printing and may be deposited by the 3D printer based on print instructions that are in accordance with a volumetric material description. The 3D printer may produce a heterogeneous and continuously varying material composite, which has tunable physical properties (e.g., water-absorbency, hydrophilicity, wetting, and diffusivity). The 3D printer may deposit chemical signaling resins and other materials in order to embed these chemical signals and a diffusion enabling (e.g., semi-water-absorbent) matrix into the solid polymer object while the latter is being fabricated.

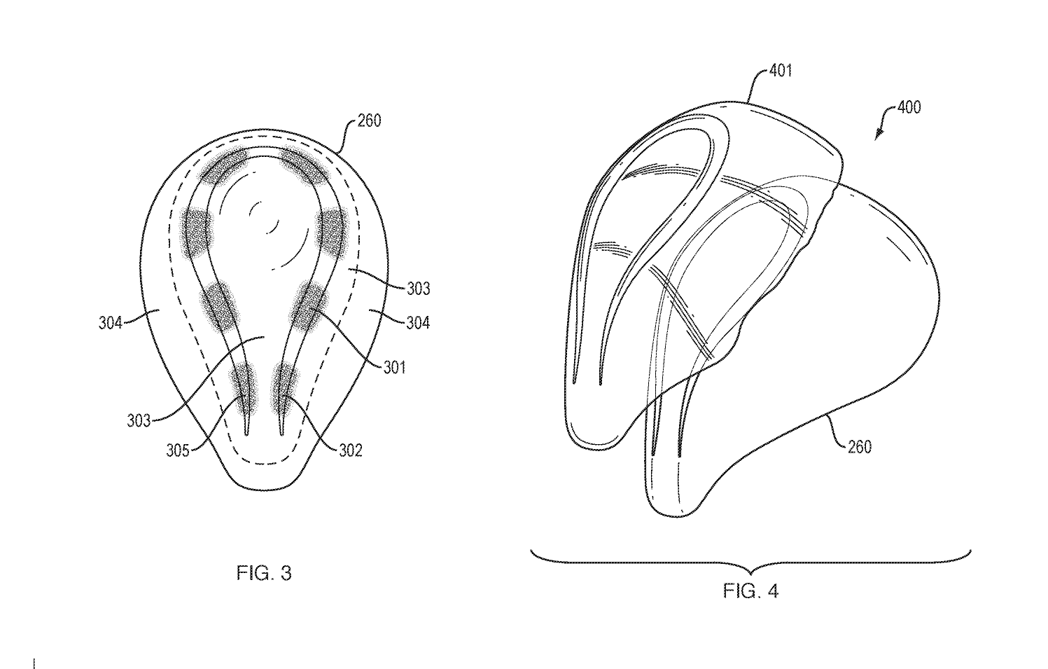

FIG. 3 shows a solid polymer structure and the position of different materials in the structure. In the example shown in FIG. 3, the solid polymer structure 260 (which has been fabricated by the 3D printer) comprises multiple polymers, including: (a) a first polymer that is located in region 304, but not in region 303, of the structure; and (b) a second polymer that is located in region 303, but not in region 304, of the structure. In addition, different chemical signals are embedded at different locations in the polymer structure during fabrication of the structure. This may be achieved by the 3D printer depositing each of the chemical signals in only certain voxels of the polymer structure. For instance, in FIG. 3, a first chemical signal is embedded at location 301, a second chemical signal is embedded at location 302, and a third chemical signal is embedded at location 305.

In some implementations, the solid polymer structure 260 is large. For instance, in some cases, the maximum dimension of polymer structure 260 is greater than 1 millimeter, or greater than 5 millimeters, or greater than 1 centimeter, or greater than 5 centimeters, or greater than 1 decimeter, or greater than 5 decimeters, or greater than 1 meter. In some cases, the maximum dimension of polymer structure 260 is greater than or equal to 1 millimeter and less than or equal to 1 meter.

In illustrative implementations: (a) a 3D printer fabricates a solid polymer structure; and (b) then a hydrogel, which contains living organisms, is applied in a thin coating that adheres consistently to the surface of the structure. For instance, the hydrogel may be sprayed onto the surface of the structure.

FIG. 4 is an exploded view of the printed, solid polymer structure 260 and hydrogel 401 that has been applied to the surface of the structure. Hydrogel 401 contains living organisms (e.g., genetically-engineered bacteria). The living organisms may be surrounded by, and distributed throughout, all or a portion of the hydrogel. Together, the polymer structure 260, hydrogel 401 and the living organisms comprise an HLM (hybrid living material) 400.

Hydrogel 401 may conform to and fit snugly against an external surface of polymer object 260. For instance, hydrogel 401 may be a thin coating over an external surface of polymer structure 260. For clarity of illustration, FIG. 4 is an exploded view in which hydrogel 401 does not appear to be touching an external surface of polymer structure 260. In actual practice, however, hydrogel 401 may touch and be in direct physical contact with an external surface of polymer object 260.

In actual practice, hydrogel 401 may be much thinner, relative to the polymer object 260, than is shown in FIG. 4. In some cases, hydrogel 401 has a thickness that is less than 3.0 millimeters, or less than 2.5 millimeters, or less than 2.0 millimeters, or less than 1.5 millimeters, or less than 1.0 millimeters. In some cases, hydrogel 401 has a thickness that is greater than or equal to 0.5 millimeters and less than or equal to 3.5 millimeters. For instance, hydrogel 401 may have a thickness of 1 millimeter, or a thickness of 2 millimeters. In some cases, the thickness of polymer structure 260 is at least ten times greater than the thickness of hydrogel 401. In some cases, the thickness of hydrogel 401 is at least ten times greater than the average diameter of the unicellular, living organisms that are surrounded by the hydrogel. In some cases, the thickness of hydrogel 401 is at least four orders of magnitude greater than the average diameter of the unicellular, living organisms that are surrounded by the hydrogel. In some cases, the thickness of hydrogel 401 is at least ten times less the thickness of polymer structure 260. In each case mentioned in this paragraph, the thickness of an object may be the maximum thickness of the object as measured along a geometric line that is parallel with and intersects a surface normal of the polymer structure 260.

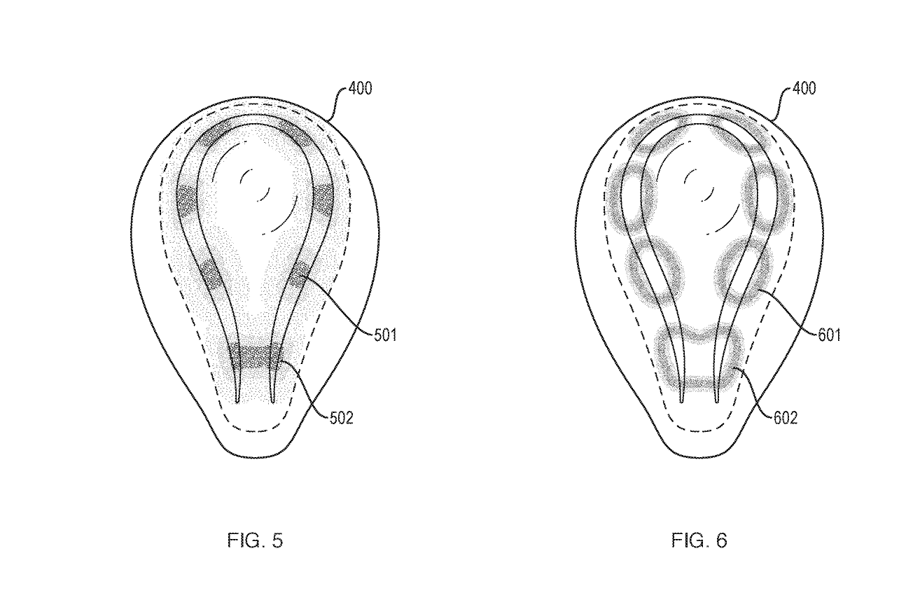

FIG. 5 shows diffusion of embedded chemicals in the HLM 400. For instance, a first chemical signal that was initially embedded in region 301 in FIG. 3 has diffused throughout region 501 in FIG. 5. Likewise, a second chemical signal that was initially embedded in region 302 in FIG. 3 has diffused throughout region 502 in FIG. 5.

Each of the embedded signals may comprise a chemical biosignal. A hydrogel that contains living, genetically-engineered single-celled organisms may be applied to a surface of the printed, solid, polymer structure. The genetically-engineered single-celled organisms may respond to the embedded biosignals as the biosignals diffuse out of the solid printed structure over time.

In illustrative implementations of this invention, the chemical signals may comprise inducers that control inducible systems in the living organisms. For instance, the chemical signals may comprise inducers that control one or more protein transcription factors (e.g., Lad, AraC, TetR) that initiate gene transcription. As the chemical signals (e.g., inducers) diffuse into and then across the hydrogel-coated surface, they may encounter living organisms (e.g., cells) genetically-encoded inducible systems, and thereby influence the gene expression levels of these living organisms.

FIG. 6 shows a programmed spatial distribution of gene expression induced by diffused chemicals. For instance, in FIG. 6: (a) gene expression that is due to the first chemical signal (which was embedded in region 301 in FIG. 3 and then diffused outward) creates a ring-shaped pattern 601; and (b) gene expression that is due to the second chemical signal (which was embedded in region 302 in FIG. 3 and then diffused outward) creates a ring-shaped pattern 602. Alternatively, multiple chemical signals may interact with each other to implement a more complicated logic with the biological signal (e.g., an AND or NAND gate

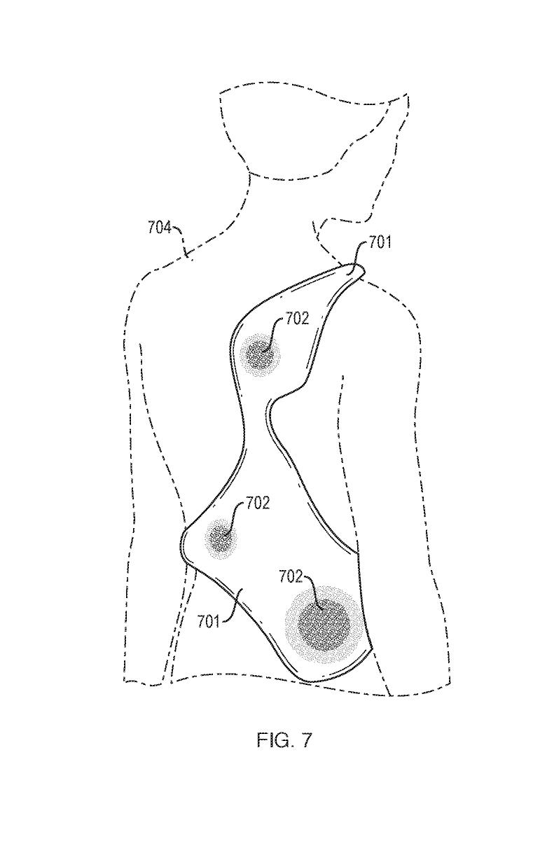

FIG. 7 shows a use case, in which a back brace induces the production, activation, or degradation of medicine by engineered biological systems, to deliver specific spatiotemporal profiles of medicine to the surface of a person's back to treat a medical condition. In FIG. 7, a chemical signal is embedded in locations 702 in a back brace 701 during 3D fabrication of the brace. A hydrogel coating containing an inducible biological system is added to the object surface. Back brace 701 may be worn on a person's back 704, while the medicine is released from the back brace in accordance with the spatial and temporal profile of gene expression controlled by the distributed inducible system, in response to chemical signals (or other stimuli). For example, the medicine may reduce pain or inflammation and be tailored to the specific patient.

In the example shown in FIG. 7: (a) back brace 701 comprises a polymer structure; (b) a hydrogel coating (not shown in FIG. 7) coats a surface of back brace 701 and also touches the person's back 704; (c) a diffusible chemical signal is initially embedded at locations 702 in back brace 701 and then diffuses into the hydrogel coating, where it regulates gene expression of living organisms in the hydrogel coating; and (d) this controlled gene expression creates products that have medicinal value and that come into contact with the person's back 704.

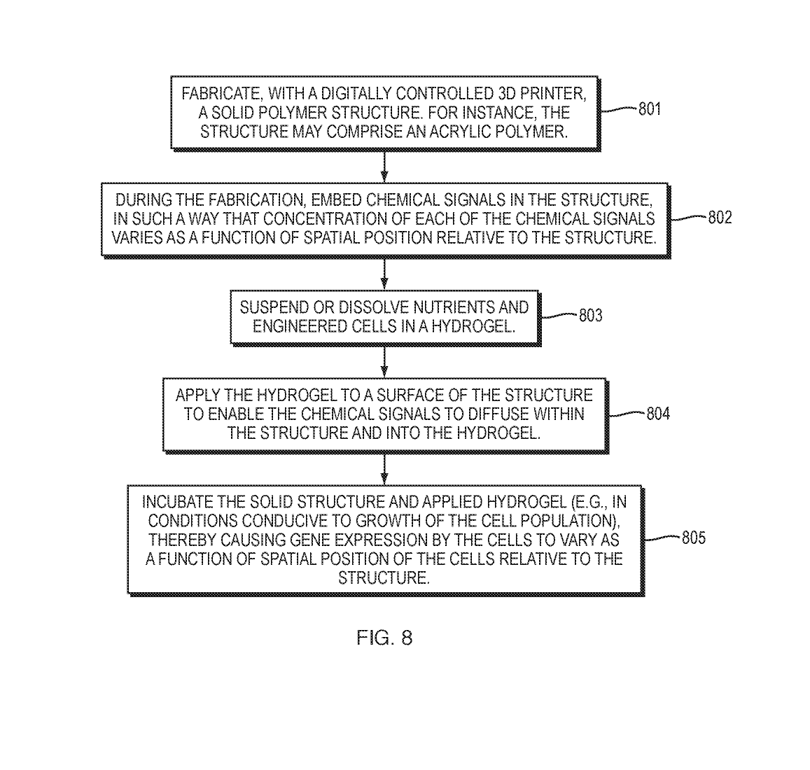

FIG. 8 is a flowchart of a method of controlling gene expression by diffusion of chemicals that are embedded in a solid polymer structure. In the example shown in FIG. 8, the method comprises at least the following steps: Fabricate, with a digitally controlled 3D printer, a solid polymer structure. For instance, the structure may comprise an acrylic polymer (Step 801). During the fabrication, embed chemical signals in the structure, in such a way that concentration of each of the chemical signals varies as a function of spatial position relative to the structure (Step 802). Suspend or dissolve nutrients and engineered cells in a hydrogel (Step 803). Apply the hydrogel to a surface of the structure to enable the chemical signals to diffuse within the structure and into the hydrogel (Step 804). Incubate the solid structure and applied hydrogel (e.g., in conditions conducive to growth of the cell population), thereby causing gene expression by the cells to vary as a function of spatial position of the cells relative to the structure (Step 805). In some implications, this results in templated products or properties (e.g. chemical, physical, visual) at the surface of the solid structure.

In some implementations, an HLM exhibits visual indicator (chromogenic enzymes and fluorometric protein) expression patterning across their surfaces due to diffusible chemical signals. The position and concentration of the diffusible chemical signals may be precisely controlled (during fabrication of a solid polymer structure that is part of the HLM), and thus the position and level of response of gene expression of a protein or RNA product of interest may be controlled with spatiotemporal precision and repeatability on the surface of 3D objects.

In some implementations, the system controls bacterial gene expression across complex geometries up to nearly half a meter in length, exhibiting varying physical properties, such as structural rigidity, localized flexibility, transparency, and color. The system may control the formation of biological patterns by orthogonal chemical signaling. The system may employ computationally controllable chemical environments to incorporate patterned bacterial behavior and product on the surface of objects with structural properties. In some implementations, tunable printing variables (i.e., geometry, material composition, signal placement/concentration) interact with engineered cell circuits to create programmable biological patterning. The spatially distributed inducible system (including engineered cell circuits) may, loosely speaking, “compute” a diffusing chemical signal.

In some implementations, living cells or inducible biological systems provide sensing and programmable computational functionality beyond patterned diffusion of chemicals alone. The engineered inducible biological systems may be programmable to be stimuli-responsive to specific single or multiple input commands. They may have the capacity to computationally process input signals, i.e. amplify signal, invert signal, create temporal functions (e.g. like an oscillator), count signals (e.g. like a state machine), or create conditionals based on neighboring agents' signals (e.g. quorum). Furthermore, any input condition may be placed in control of any function, such as the creation of a protein or RNA, the synthesis metabolization of a compound, or the modulation of enzymatic activity.

In some implementations, the regulation of gene expression results in useful biological products or reactions (such as proteins, RNA, enzymes or enzymatic reactions) being produced or caused in a spatial pattern in the hydrogel coating.

In some cases, the living organisms (e.g., cells) are kept alive and functionality of the HLM depends on their continued biological responsiveness. For instance, the continued responsiveness of the living organisms may enable periodic temporal expression patterns, or multi-input circuits that also respond to external or environmental stimuli.

Alternatively, in some implementations, the living organisms perish or desiccate after the gene expression process, leaving behind a deposition or result of expression products (i.e. a pigment, a chemical product, a surface modification such as mineralizing or texturizing solid structure surface). Thus, in some cases, a product (which is produced as a result of the gene expression) persists after the death or desiccation of the living organisms.

In some implementations, a computer algorithm employs a computational model for simulation of HLM biological response—based on surface geometry, diffusivity, and one or multiple gene response profiles—as a tool for the design and fabrication of HLM constructs with predictable and repeatable gene expression patterning.

In some implementations, hybrid living materials (HLMs) combine living and non-living components to harness the biological capabilities of organisms within structural materials.

### Hardness

In some cases, all of part of the solid polymer structure is hard. For instance, all or part of the solid polymer structure may have a hardness, on Shore Durometer Scale D (a scale for harder materials), that is (a) greater than or equal to 80; or (b) greater than or equal to 80 and less than or equal to 88. Alternatively or in addition, all or part of the solid polymer structure may be softer. For instance, all or part of the solid polymer structure may have a hardness, on Shore Durometer Scale A (a scale for softer materials), that is (a) greater than or equal to 20; (b) greater than or equal to 20 and less than or equal to 77; or (c) greater than or equal to 4 and less than or equal to 20. For example, a flexible or semi-absorbent portion of the solid polymer structure may have a hardness, on the Shore Durometer Scale A that is equal to 10. The semi-absorbent portion of the polymer structure may be able to absorb at least a limited quantity of water. The 3D printer may control the hardness/softness of each voxel of the solid, polymer structure by depositing different mixes of materials (e.g., resins or other polymers) in different spatial positions while fabricating the structure.

In some cases: (a) all or part of the solid polymer structure is hard and rigid; and (b) this hardness and rigidity enables the polymer structure to be a weight-bearing, permanent, structural framework for the HLM.

### Smoothness, Lack of Porosity

In some cases, the solid polymer structure has a smooth external surface. For instance, in some cases, a region of an external surface of the solid polymer structure: (a) is at least one square millimeter in area; and (b) has a surface roughness that is greater than or equal to 0.8 microns R_{α} and less than or equal to 1.2 microns R_{α}. In some cases, the solid polymer structure: (a) is at least one square millimeter in area; and (b) does not have any feature that deviates in height by more than 1 micron from the mean height of the region.

In some cases, the solid polymer structure does not have any pores or internal cavities (or has only pores or cavities that are smaller than the single-celled organisms in the hydrogel). Here are some non-limiting examples:

In some cases, at least one region of the solid polymer structure: (a) is a rectangular cuboid; (b) has a volume that is greater than or equal to 1 microliter; (c) does not have any internal cavity that is greater than 1 micron in diameter; and (d) does not have any pore that is greater than 1 micron in diameter.

In some cases: (a) at least one region of the solid polymer structure is a rectangular cuboid and has a volume that is greater than or equal to 1 microliter; (b) the average diameter of the cavities, if any, in the region is less than 1 micron; and (c) the average diameter of the pores, if any, in the region is less than 1 micron.

In some cases, at least one region of the solid polymer structure: (a) is a rectangular cuboid; (b) has a volume that is greater than or equal to 1 microliter; (c) does not have any internal cavity that is greater than 100 nm in diameter; and (d) does not have any pore that is greater than 100 nm in diameter.

In some other cases: (a) at least one region of the solid polymer structure is a rectangular cuboid and has a volume that is greater than or equal to 1 microliter; (b) the average diameter of the cavities, if any, in the region is less than 100 nm; and (c) the average diameter of the pores, if any, in the region is less than 100 nm.

In some cases: (a) the 3D printer deposits liquid resin droplets that spread before they are cured by UV light; and (b) this spreading of the liquid droplets causes the solid polymer object to have a smooth surface and to not have any macro- or micro-scale (i.e. 1 micron scale, or larger) pores or internal cavities.

### Photopolymers

In some cases, the 3D printer deposits acrylic or acrylic-based polymers (or monomers) to fabricate the solid structure.

In some cases, the deposited materials include photopolymers. After being deposited by the 3D printer, photopolymerizable resins may be cured by being exposed to light.

For instance, the photopolymers may comprise polymer compositions that contain a photo-initiator or photo-initiator system. For instance, an acylphosphine oxide type photo-initiator may be employed. The deposited photopolymers may be exposed to light (e.g., UV light) and may undergo photolytic/UV-light-induced polymerization (e.g., where the photopolymerization comprises a Norrish Type I reaction or Norrish Type II reaction).

In some cases, the deposited materials include low viscosity resins. For instance, the low viscosity resins may have a viscosity of less than 100 millipascal-seconds at 25 degrees Celsius. The low viscosity may facilitate deposition (e.g., jetting from an inkjet printer). To achieve a low viscosity, a low-viscosity reactive diluent may be employed (e.g. an N-vinyl monomer, or an acrylate oligomer such as DPGDA (diacrylate) or PETA (tetraacrylate)).

In some cases, the 3D printer deposits polymer compositions that include one or more of the following: acrylate-based monomers, acrylate-based oligomers (small chains, e.g. diacrylate, dimethacrylate), and acrylate solutions (e.g. liquid acrylate, liquid methacrylate).

In some cases, the 3D printer deposits multiple acrylate-based polymer compositions with varying degrees of post-cured water-related properties (e.g., absorbency, diffusivity, hydrophilicity, wetting) while fabricating a single solid structure. The monomers and polymers may be included in the polymer networks and cause these networks to have different properties (e.g., absorbency, diffusivity, hydrophilicity, wetting) with respect to water or solutions of varying pH.

In some cases, the 3D printer performs on-demand blending (before curing) of multiple resins to create a continuous gradient of compositions that have intermediate levels of absorbency, and thus to achieve polymers that accept varying amounts of water-based solutions into their networks. These polymers may contain enough non-water-absorbent components that they have sufficient strength to remain as permanent parts of the printed, solid structure (rather than being sacrificed or dissolved after the structure is printed).

In some cases, the polymer compositions that are deposited by the 3D printer contain chemical additives. The solid structure's capacity to have semi-water-absorbent properties in conjunction with chemical additives may enable the polymer network to store chemicals upon curing, and may enable the polymer network to release these chemicals upon exposure to a water-based environment.

The level of water-miscibility ((e.g. a property enabling chemical signal diffusion) may be specified on a voxel-by-voxel basis in a CAD (computer-aided design) model. Digital print instructions for the 3D printer may be generated based on this CAD model. Thus, the water-miscibility of the solid structure that is fabricated by the printer may be programmatically controlled.

In some cases, one or more material compositions deposited by the 3D printer each include at least one reactive component, at least one photo-initiator, at least one surface-active agent and at least one stabilizer. For instance, the photo-initiator may comprise a free radical photo-initiator, a cationic photo-initiator, or any combination thereof. The reactive component may comprise an acrylic monomer, an acrylic oligomer, and or an acrylic crosslinker, or any combination thereof. In some cases, the reactive component comprises at least one water-absorbent component that, after curing, swells upon exposure to water or to an alkaline or acidic water solution. The water-absorbent component may comprise acrylated urethane oligomer derivative of polyethylene glycol, a partially acrylated polyol oligomer, an acrylated oligomer having hydrophilic substituents, or any combination thereof. In some cases, the hydrophilic substituents are acidic substituents, amino substituents, hydroxy substituents, or any combination thereof.

In some cases, the materials deposited by the 3D printer include a non-reactive component such as polyethylene glycol, methoxy polyethylene glycol, glycerol, ethoxylated polyol, or propylene glycol.

In some cases, the 3D printer deposits a photopolymer composite that includes one or more of VeroClear™ and SUP705™.

The VeroClear™ may include acrylic monomers, acrylic oligomer(s), a photo-initiator, and camphene (CAS No. 79-92-5). For instance, the acrylic monomers may include: (a) 4-(1-Oxo-2-propenyl)-morpholine (CAS No. 5117-12-4); (b) isobornyl acrylate (CAS No. 5888-33-5); (c) acrylic acid ester; and (d) 2-Propenoic acid (CAS No. 79-10-7). The acrylic oligomer may comprise phenol, 4,4′-(1-methylethylidene)bis-, polymer with (chloromethyl)oxirane, 2-propenoate. The photo-initiator may comprise diphenyl-2,4,6-trimethylbenzoyl phosphine oxide.

The SUP705™ may include non-reactive components, an acrylic, a stabilizer and a photo-initiator. The non-reactive components may be the primary ingredients and may include water-absorbent components, such as polyethylene glycol 400 (PEG), propylene glycol (PG, propane-1,2-diol) and glycerol, 1,2,3-propanetriol (CAS No. 56-81-5) soluble. The acrylic may comprise acrylic acid, 2-hydroxyethyl ester, 2-hydroxyethyl acrylate (CAS No. 818-61-1). The stabilizer may comprise mequinol, 4-methoxyphenol.

More generally, in some cases, the 3D printer deposits acrylic compounds that are combined with a photo-initiator and cured through exposure with UV light. Thus, the materials that are deposited may include isobornyl acrylate (CAS number 5888-33-5), acrylic acid ester (CAS number 52408-84-1), acrylic monomer(s) and acrylic oligomers. In some cases, the materials listed in the preceding sentence are mixed, before being deposited, with one or more of: (a) phosphine oxides; (b) diphenyl(2,4,6-trimethylbenzoyl) (CAS number 75980-60-8); (c) glycerin (CAS number 56-81-5); (d) 1,2 propylene glycol (CAS number 57-55-6); and (e) polyethylene glycol (CAS number 25322-68-3)

In illustrative implementations, the 3D printer does not deposit living cells. Because the uncured polymer compositions that are deposited by the 3D printer do not contain living cells, they may be cytotoxic while they are uncured.

In many implementations, the deposited materials (and the solid structure that results from curing the deposited materials): (a) are not resorbable and are not biodegradable by the human body; (b) are not replaced, after being printed, by biological materials produced by living cells; (c) are not porous on a macro- or micro-scale, and (d) are not infiltrated by living cells.

### Chemical Signals/Inducers

In some cases, chemical signals are deposited by the 3D printer during fabrication of the solid polymer object. Thus, the chemical signals may be embedded in the solid polymer object at the time of fabrication. After being fabricated, the solid polymer object may be exposed to moisture or water (e.g., water in a hydrogel). The embedded chemical signals may then diffuse through and then out of the solid object, and into a hydrogel that is adjacent to the solid object and that contains living cells. The living cells may be genetically engineered in such a way that one or more chemical agents control or regulate gene expression in the cells. The gene expression that is conditionally induced may comprise synthesis of protein or RNA (ribonucleic acid).

Each chemical signal may comprise a diffusible chemical additive embedded in the polymer structure.

In some cases, each chemical signal comprises an inducer. Specifically, each chemical signal may comprise one or more chemical or small molecule stimuli that are a specific exogenous or endogenous effector (i.e., inducer) of a genetically-encoded, inducible system. The inducible system may have been placed in control of recombinant gene(s) of interest, within cells or biological systems that populate the hydrogel in the HLM. Each chemical signal may function as an inducer to a corresponding inducible system that has been engineered within the incorporated living cells. The inducible systems may control the levels of a gene expression in response to one or more of the chemical signals. These inducible systems may comprise one or more protein transcription factors (e.g., Lad, AraC, TetR) whose ability to initiate gene transcription, and hence gene expression—often, by binding to region of DNA within the gene's promoter—is dependent on the presence or concentration of a given inducer (e.g. IPTG, arabinose, tetracycline respectively).

In some cases, to create the genetically engineered cells, recombinant genes are assembled that place inducible systems “in control” of a biosynthetic product or output of interest. The inducible systems may exhibit increased sensitivity and dynamic range towards a specific inducer (e.g., chemical signal embedded in the polymer structure), and lower crosstalk or background.

In illustrative implementations, each diffusible chemical signal that is embedded in the polymer is an inducer. The inducer may have one or more of the following five characteristics: (1) neither the print process (UV- or thermal-exposure) nor the polymer chemistry significantly degrade, disable, or negatively affect the biological functionality or recognition of the molecule; (2) directly, or by way of the solvent it resides in, the inducer molecule does not affect the viscosity of the resin beyond the window acceptable for jetting; (3) the inducer molecule may be stably suspended or located within a cured polymer network; (4) upon addition of an aqueous environment to the surface of the cured polymer structure, the inducer readily diffuses out of some (not necessarily all) of the possible cured polymeric network compositions, thus becoming independently distributed across the 3D surface layer of the structure; and (5) the inducer interacts with one or more cognate recombinant gene expression systems, by driving the inducible system towards the up- or down-regulation of a controlled gene.

In some cases, chemical substrates are incorporated into the polymer structure in order to functionally interact with the product of the regulated gene constructs. For instance, a chemical may induce the up regulation of an enzyme that in turn acts upon a diffusing chemical substrate at distinct spatial locations. For example, the method described in the preceding sentence may be applied to create a drug, or the precursor to a useful compound, or a visual indicator.

In some cases, one or more of the chemical signals in the polymer structure comprises an AHL (acylated homoserine lactone). These AHL chemical signals may comprise quorum sensing inducers for bacterial systems. In some cases, this enables adjacent intracellular communication, global mechanisms or spatiotemporal synchronicity (e.g. edge detection functions, globally-gated moratoriums or responses, or periodic oscillations, respectively).

In some cases, the chemical signals (i.e. inducers) that are embedded in the polymer structure are inducers for bacterially-derived inducible systems that control synthetic gene circuits. For instance, the chemical signals (inducers) for the bacterially-derived inducible systems may comprise one or more of the following: (a) isopropyl β-D-1-thiogalactopyranoside (IPTG) (to induce expression that is under the control of Plac or derivative, such as PTac or PLlacO); (b) rhamnose (Rham) (to induce expression that is under the control of PRha); (c) 3-hydroxytetradecanoyl-homoserine lactone (OHC14, AHL-class) (to induce expression that is under the control of cinR or Pcin); (d) 3-oxohexanoyl-homoserine lactone (OC6, AHL-class); (e) 2,4-diacetylphophloroglucinol (DAPG) (to induce expression that is under the control of phlF or PPhlF); (f) cuminic acid (Cuma) (to induce expression that is under the control of cymR or PCymRC); (g) vanillic acid (Van) (to induce expression that is under the control of vanR or PVanCC); (h) anhydrotetracycline (aTc); (i) L-arabinose (Ara) (to induce expression that is under the control of araC/araE or PBAD); (h) choline chloride (Cho) (to induce expression that is under the control of Betl or PBetl); (j) naringenin (Nar) (to induce expression that is under the control of ttgR or PTtg); (k) 3,4-dihydroxybenzoic acid (DHBA) (to induce expression that is under the control of pcaU or P3B5B), and (1) sodium salicylate (Sal) (to induce expression that is under the control of nahR or PSalTTC).

Alternatively or in addition, the chemical signals (inducers) in the polymer structure comprise one or more of the following: (a) amino acids; (b) amino acid analogs; (c) saccharides; (d) polysaccharides, (e) nucleic acids; (f) protein transcriptional activators or repressors; (g) cell signaling molecules; (h) toxins; (i) petroleum-based compounds; (j) metal containing compounds; (k) salts; (l) enzyme substrate analogs; and (m) hormones.

In some cases, organisms are immobilized in the hydrogel and function as genetically-encoded “sensors” for small molecules. For instance, these organisms may be genetically engineered in such a way that: (a) they contain a foreign metallothionein gene (e.g., from yeast, mammal genetic material, able to bind or sequester metal ions) and (b) expression of this metallothionein gene is controlled by small molecule inducers (e.g., metals) that have diffused from the polymer structure, the external environment, or other embedded cells or biological systems.

In some cases, the hydrogel includes chemical substrates, as in compounds upon which the products of the regulated gene expression (e.g., enzymes) may act to produce chemical products. For instance, in some cases, the hydrogel includes one or more of the following substrates that are acted upon by β-galactosidase:(a) 5-bromo-4-chloro-3-indolyl-β-D-galactopyranoside; (b) 6-Chloro-3-indolyl-β-D-galactopyranoside; (c) 5-Iodo-3-indolyl-β-D-galactopyranoside; (d) 5-Bromo-6-chloro-3-indolyl-β-D-glucopyranoside; (e)N-Methylindolyl-β-D-galactopyranoside; and (f) 4-Methylumbelliferyl β-D-galactopyranoside. Likewise, the hydrogel may include indoxyl glycosides (or esters) that yield insoluble pigments in response to B-galactosidase activity.

In some implementations, the genetically engineered organisms in the hydrogel include inducible promoters that are regulated by inducers. The promoters may comprise a region of a nucleic acid sequence at which initiation and rate of transcription of the remainder of a nucleic acid sequence are controlled. Each of these promoters may drive expression or may drive transcription of the nucleic acid sequence that it regulates. These promoters may be inducible—that is, may initiate or positively (enhance) or negatively (repress) modulate transcriptional activity when in the presence of an inducer. The inducers may comprise chemical signals that: (a) are initially embedded in, and then diffuse out of the 3D printed, solid structure; and (b) then diffuse into the hydrogel in which the organisms are immobilized.

In some cases, the inducible systems in the hydrogel are responsive to external stimuli in conjunction with chemicals and small molecule inducers, in multi-input or combinational logic circuits for gene regulation.

In some cases, the chemical signals (which are initially embedded in, and then diffuse of, the 3D printed, solid structure) comprise one of more ligands, bioactive agents, regulatory signals, biosignals, or biochemicals.

### Hydrogel

In illustrative implementations, hydrogels are applied to the surface of the solid polymer, polymer structure. The hydrogel may surround the genetically engineered living organisms. The hydrogel may include nutrients and growth media and may provide a biocompatible environment in which the organisms may thrive and multiply.

The hydrogels may: (a) comprise polymer networks that are extensively swollen with water; an (b) function as a thin, hydrophilic or aqueous coating that adheres consistently across the surface of the polymer structure. Water in the hydrogel may facilitate the diffusion of chemical signals from the solid polymer structure to the living organisms in the hydrogel. Among other things, water from the hydrogel may move into the solid polymer, polymer structure, thereby enabling or facilitating the diffusion of chemical signals out of the solid structure and into the hydrogel.

The hydrogel may substantially immobilize the living organisms (relative to the hydrogel and 3D printed structure) thereby facilitating gene regulation of the living organisms that is independently controllable in different regions of the hydrogel. This spatially controllable gene regulation may be achieved because: (a) the initial distribution of the chemical signals in the solid polymer structure is controlled on a voxel-by-voxel basis; (b) this initial distribution thus determines the spatial diffusion pattern of the chemical signals from the solid printed structure into the hydrogel and then inside the hydrogel; and (c) the living organisms are substantially immobilized in the hydrogel, and thus the spatial diffusion pattern of the chemical signals determines which concentration of chemical signals affect each respective living organism in the hydrogel.

The hydrogel may create and maintain an aqueous environment, by which living cells or biological systems: (a) are viable on the surface of the polymeric structure; and (b) are able to carry out natural or recombinant cell/biological function.

The hydrogel may immobilize living cells or biological systems across the surface of the polymer structure, to create a multicellular sensing construct out of multiple cells that may be loosely described as “genetically-encoded sensors”.

The hydrogel may create and maintain an aqueous environment by which diffusive chemical signals, from the polymer structure, the external environment, or other embedded cells or biological systems may interact with the inducible system, e.g., an aqueous transport medium.

In some cases, the hydrogel's internal network is formed by thermal-dependent physical bonds (e.g. hydrogen bridges). The solidification temperature of the hydrogel may be less than the maximum temperature at which the living organisms may thrive and proliferate.

In some cases, the hydrogel includes agar, agarose, and water. Both agar and agarose are linear polysaccharides, which, when dissolved in water, form a gel when cooled to at least room temperature.

The hydrogel may comprise a so-called chemical hydrogel, with an internal network formed by chemical bonds. Alternatively, the hydrogel may comprise a so-called physical hydrogel: i.e., have an internal network that results from physical bonds (e.g., physical intermolecular associations). Alternatively, the hydrogel may have an internal network formed by crystallites or other junctions that remain intact within the extending fluid.

In some cases, the hydrogel is a physical hydrogel that includes any combination of one or more of the following materials: polysaccharides, agar, agarose, starch, alginate, chitosan, dextran, carrageenan, proteins/protein matrices, gelatin, collagen, cartilage, fibrin, poly(lactic acid) (PLLA), hyaluronic acid, carboxymethylcellulose, peptide amphiphile-ti composite, and chondroitin sulfate.

In some cases, the hydrogel may comprise a chemical hydrogel (also known as an irreversible hydrogel) which has an internal network formed largely by crosslinking. In some cases, where a chemical hydrogel is employed: (a) the polymer or monomer components that are precursors to crosslinking are biocompatible; or (b) the chemical crosslinking reaction is also biocompatible.

In some cases, the hydrogel includes one or more of the following materials: poly(ethylene glycol) (PEG); poly(vinyl alcohol) (PVA); poly(acrylic acid) (PAA); poly(hydroxyl ethyl methacrylate) (PHEMA); diacrylated (DA) or dimethacrylated (DMA) derivatives of PEG; acrylamide or polyacrylamide; and sodium acrylate.

In some cases, the hydrogel comprises a combination of one or more physical gels and one or more chemical gels, or additives that comprise a “tough hydrogel”. In some cases, the hydrogel comprises: (a) polyacrylamide-alginate (PAAm-alginate) hydrogel; (b) fiber-reinforced tough hydrogel; or (c) elastomer-bonded tough hydrogel.

In some cases, the hydrogel includes superabsorbent or superporous hydrogel components, such as acrylamide, acrylic acid, or salts of acrylic acid (e.g., sodium, sulfopropyl, and potassium acrylates) or 2-hydroxyethyl methacrylate.

In some cases, the hydrogel includes two or more components with differing physical or functional properties. For instance, a top layer to retain water content/prevent evaporation, or provide a semipermeable barrier.

In some implementations of this invention, a hydrogel-elastomer composition encapsulates a population of genetically-engineered cells, where each of the cells contains a promoter operably linked to a nucleic acid encoding a product of interest.

### Living Organisms

In some cases, the living organisms (e.g., cells) in the hydrogel are genetically-engineered. The living organisms in the hydrogel may comprise an inducible system (e.g., a promoter operably linked to a nucleic acid encoding a product of interest) that corresponds to an inducer embedded in the polymer structure.

In some cases, the living organisms in the hydrogel comprise single-celled organisms, such as bacterial cells (e.g., E. coli or B. subtilis), single-celled prokaryotes, single-celled eukaryotes (e.g. Saccharomyces ssp., a type of yeast), extremophiles, archaea (e.g., Halobacteriaceae ssp.), algae, or spores of bacteria or fungi.

In some cases, the living organisms in the hydrogel comprise multi-cellular organisms or multi-cellular systems. Each multi-cellular system, or units thereof, may be surrounded by hydrogel and may respond to diffusing chemical signal in a localized way, relative to the spatial regions of the polymer structure.

In some cases, the hydrogel contains a biological system that (a) does not consist of whole cells and (b) comprises a gene-encoded sensor, inducible system or both. For instance, the hydrogel may contain a “cell free” system that functions as an inducible system (e.g. regulated translation, protein synthesis). The cell-free system may be cell extract-based or purified enzyme-based. In some cases, the hydrogel includes viruses or DNA with a supporting biological system (e.g. ribosomes) to biosynthesize the product or output of interest.

In some cases, the immobilization of many gene-encoded sensors across the surface of the 3D-printed polymer structure helps create a multi-sensor construct (e.g., a multicellular sensing construct) across the surface of the 3D-printed polymer structure. This multi-sensor construct may respond to specific signals which diffuse from the polymer structure, and thus produce patterned outputs that alter the polymer surface selectively and in a programmed manner.

With genetically-encoded logic, the multi-sensor construct may implement spatially distributed computation or synthetic consortia.

In some cases, the genetically-engineered cells in the hydrogel comprise: prokaryotic cells (e.g., bacterial cells); or eukaryotic cells (e.g., yeast or mammalian cells).

In some cases, the genetically-engineered cells in the hydrogel include a genetic circuit which comprises: a promoter operably linked to a nucleic acid encoding a product of interest (e.g., a nucleic acid or protein of interest).

In some cases, the genetically-engineered cells in the hydrogel comprise bacterial cells, such as any of the following: Yersinia spp., Escherichia spp., Klebsiella spp., Acinetobacter spp., Bordetella spp., Neisseria spp., Aeromonas spp., Franciesella spp., Coryne bacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Streptomyces spp., Bacteroides spp., Prevotella spp., Clostridium spp., Bifidobacterium spp., or Lactobacillus spp.

In some cases, the genetically-engineered cells in the hydrogel comprise bacterial cells, such as any of the following: Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Coryne bacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus cereus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. In some embodiments, the cells are Escherichia coli cells.

In some cases, the genetically-engineered cells in the hydrogel comprise mammalian cells.

In illustrative implementations of this invention, the genetically engineered, living organisms in the hydrogel may contain recombinant DNA and may produce (as a result of expression of the recombinant DNA) recombinant proteins or functional RNA. The recombinant DNA may be created by molecular cloning. The recombinant DNA may have been introduced into an ancestor of the living organisms by: (a) transformation (e.g., passing foreign DNA through cell walls of bacteria); (b) transfection (e.g., inserting foreign DNA into animal cells), or (c) transduction (e.g., introducing foreign DNA into a cell via a virus or viral vector). For instance, if transfection is employed, the transfection may be facilitated by a solution that contains calcium phosphate and foreign DNA.

In some cases, genetic editing is performed to introduce or remove DNA from the genetically-engineered cells that are in the hydrogel. The genetic editing may introduce foreign DNA into a cell. The genetic editing may employ artificially engineered nucleases that create double-stranded breaks at precise locations in DNA. For instance, the engineered nucleases may comprise: (a) meganucleases; (b) zinc-finger nucleases (ZFN); (c) transcription activator-like effector nucleases (TALEN); or (d) clustered regularly interspaced short palindromic repeat/CRISPR associated protein (CRISPR/Cas). For example, the CRISPR/Cas may comprise CRISPR/Cas9.

### Nutrients

In some cases, the nutrients in the hydrogel include: (a) a source of carbon, (b) salts (e.g. salts that contain magnesium, nitrogen, phosphorus, or sulfur) to allow the bacteria to synthesize protein and nucleic acids, or to activate enzymes; (c) amino acids; (d) vitamins or (e) trace elements. They may also include a selective pressure (e.g. antibiotic) to ensure that only selected, intended microorganisms grow.

In some cases, the nutrients in the hydrogel include lysogeny broth. The lysogeny broth may include one or more of the following materials: tryptone, yeast extract, and NaCl.

In some cases, the nutrients in the hydrogel comprise a minimal media. The minimal media may include one or more of the following materials: M9 salts, Na2HPO4, KH2PO4, NH4Cl, NaCl, carbon sources, glucose, sucrose, glycerol, succinate, minerals, MnSO4, CaCl2, ZnSO4, CuSO4, CoCl2, (NH4)6Mo7O24, citamins, p-aminobenzoic acid, niacinamide, DL-pantothenic acid, calcium salt, pyridoxal HCl, pyridoxamine (HCl)2, pyridoxine HCl, riboflavin, thiamine HCl, d-biotin, folic acid, purines and pyrimidines, adenine sulfate, uracil, guanine, xanthine, amino acids, L-Alanine, L-Arginine, L-Asparagine, L-Aspartic acid, L-Cysteine, L-Glutamic acid, Glycine, L-Histidine, L-Isoleucine, L-Leucine, L-Lysine, DL-Methionine, L-Phenylalanine, L-Proline, L-Serine, L-Threonine, L-Tryptophan, L-Tyrosine, L-Valine, MgSO4, FeSO4, casamino acids, and yeast extract.

### Prototype

The following 38 paragraphs describe a prototype of this invention.

In this prototype, a multi-material 3D inkjet printer Stratasys® Objet® Connex™ 500 is employed as a digital fabrication tool. This printer is configured to print at high resolution, control multiple materials, and create complex self-supporting structures. This printer is a polymeric inkjet printer that prints a wide range of materials properties by using an array of print heads to (free-form) deliver droplets of numerous photopolymer resins (up to 14), simultaneously, to targeted positions within a macroscale build space (30×30 cm). This printer blends loaded materials on-the-fly to create expansive range of material properties.

In this prototype, the printer employs a bitmap-based printing approach or voxel printing approach. A digital file of a 3D object is converted into a set of layered Z-slices at the native height resolution of the printer, 16 μm. Each slice is represented as a bitmap file in which each pixel is an individually addressable digital material assignment, for the printer to deposit in a layer-by-layer fashion. The material assignments prescribe specific resin combinations for building absorptive materials as matrices to hold chemical signals. The printer is configured to deposit consistent droplet volumes (12 pL) of material at a high level of spatial accuracy. This spatial precision is used to precisely distribute chemical signal throughout the build volume of the 3D structure. The print description is optimized for absorptive material properties and chemical signal gradients.

In this prototype, the 3D printer is configured to produce structures of up to 0.49 meters in length or 38 liters build volume (49×39×20 cm). The object that is fabricated may be smooth and non-macroporous. The 3D printer has CAD-controlled geometric feature resolution of down to 0.1 mm throughout.

In this prototype, the materials that are printed by the printer include UV-curable acrylate-based polymer resins that range in composition and cured material behavior. In a test of this prototype, three print resins—two traditional “build materials”, rigid VeroClear™ (RGD810) and flexible Tango™ (FLX930), and one hydroscopic swelling material (SUP705™)—were tested for suitability as a bioactive substrate. (SUP705™ is conventionally employed as a so-called “support material”. However, in many implementations of the present invention, SUP705™ is employed for other purposes). Wettability and hygroscopy tests—properties related to internal crosslinking density—identified that SUP705™ compositions were capable of encapsulating and releasing chemical signal solutions. Specifically, wetting behavior was characterized as a contact angle measured with the Sessile Drop method, and hygroscopic swelling was measured by change in weight and volume of each cured polymer over a 24-hour soak in water. Resin mixes with SUP705™ content exhibited up to a tenfold decrease in wetting angle and a 2-fold greater swelling equilibrium by weight than build material counterparts, making them well suited for the absorption and release of liquids, and thus candidate materials for embedding chemical signal solutions into printed objects.

In a test of this prototype, E. coli cured material disks were introduced to early log-phase liquid culture and incubated for 24 hours. A LIVE/DEAD assay was performed on the cells and quantified through fluorescence activated cell sorting (FACS). The assay verified E. coli viability (<50% of population is dead/injured) for all material compositions, but showed better compatibility (<15% of population is dead/injured) for SUP705™-build material intermediates than pure SUP705™. These findings indicate that composites created by unconventionally blending SUP705™ into build materials provide suitable substrates for cell culture, while creating an absorptive matrix to store and release chemical signals for the purpose of inducing gene expression. By modulating the concentration of SUP705™ in the deposited material, the printer platform controls the amount of chemical signal per location.

In a test of this prototype: (a) 50 mm diameter VeroClear™ RGD810 disks with 10 mm diameter templated centers of varying SUP705™/RGD810 compositions were printed, soaked in an IPTG-X-gal solution, and coated with a hydrogel suspension of E. coli expressing β-gal (blue); and (b) the ratio of SUP705™ per disk correlated with the saturation and localization of the blue pigment.

In this prototype, chemical signals are introduced to printed parts using one of two approaches. In a first approach, chemicals are introduced into a printed object after an object is printed. This first approach leverages the fact that SUP705™ may selectively absorb a chemical solution. In this first approach, printed parts are soaked in a H₂O/DMSO chemical signal solution for 12 hours. Isopropyl β-D-1-thiogalactopyranoside (IPTG) and the colorless reagent 5-bromo-chloro-3-indoyl-β-D-galactopyranoside (X-gal) are used for the soak, together with E. coli cells transformed with pUC19 plasmids. When the printed object is removed from the bath, only areas containing SUP705™ retains chemical signals. In a second approach, chemical signals are incorporated directly into the objects during the printing process using resins, eliminating the soaking step and allowing for multiple chemicals to be placed simultaneously. In both the first and second approaches, the absorptive matrix created by patterning SUP705™-intermediates plays a role in controlling the release of chemical signals from the cured structure.

Here is an example of the first approach (in which a rigid structure is soaked after being printed, in order to introduce chemical signals). A post-printing soak step introduces small molecules to the printed plastics. 3D printed pieces are submerged in an aqueous mixture of inducer molecule, IPTG, and a histochemical substrate, 5-bromo-4-chloro-3-indolyl-β-D-galactoside (X-gal), for 12 hours at 4° C. The soaking solution is created by dissolving powder X-gal (VWR, 20 mg/ml) and IPTG (VWR, 24 mg/ml) in DMSO. The solution is then diluted with deionized water (1:1 ratio) on ice, for a resulting concentration of 50 mM IPTG, 25 mM X-gal. After soaking, the 3D printed templates are removed from the bath and spray-coated with pUC19 E. coli suspended in hydrogel. This application of cells and media achieves even culturing, within a ˜1-2 mm thick agar layer, across the printed shapes. The growth media mixture is based on M9 minimal media (Teknova®), that is supplemented with 0.8% sucrose (VWR), 0.1% Casamino acids (VWR), 1% agar (Bacto™ Agar) and 1% agarose (VWR). The minimal media mixture is melted at 100° C. and then cooled to 45° C. prior to the addition of pUC19 E. coli overnight stock (1:200) and ampicillin. The mixture is stirred vigorously for even distribution before solidification, and transferred into a 50 mL glass bottle with a fine mist sprayer (Vivaplex®, 50 ml amber glass bottle). Each piece is spray-coated with 15 spray strokes from the mist sprayer. After coating, the pieces are put into 90 mm polystyrene culture dishes, sealed with Parafilm (Bemis NA), and incubated for 30 hours at 37° C.

In some tests of this prototype, an Escherichia coli K12 strain derivative (NEB Turbo, F′ proA+B+lacIq ΔlacZM15/fhuA2 Δ(lac-proAB) glnV galK16 galE15 R(zgb-210:Tn10)TetS endA1 thi-1 Δ(hsdS-mcrB)5) were used as the host strain. Cells were cloned via New England Bio Labs High Efficiency Transformation protocol with pUC19 Vector (NEB), containing Ampicillin resistance and reporter gene lacZ for Isopropyl β-D-1-thiogalactopyranoside (IPTG) regulated beta-galactosidase (β-gal) production. Single colonies were selected and grown in an overnight culture of LB supplemented with 100 μg/mL ampicillin (LB-Amp), at 37.5° C. Plasmids containing circuits for AND NAND logic were cloned using the Gibson method onto a p15A plasmid containing a kanamycin resistance marker. Both circuits contain the sicA/invF two-component system as a single-layer AND gate. Inversion of signal in the NAND gate is performed with the Ph1F repressor.

In some use cases of this prototype, E. coli strains perform local logic in response to the chemical signal micro-environments programed into the printed objects. For instance, the following gene constructs may be engineered to produce visual outputs (colorimetric, fluorescent) to spatially and temporally observe gene expression levels.

In some use cases, this prototype facilitates colorimetric assays of gene expression as follows: E. coli cells are transformed with the pUC19 plasmid to express β-galactosidase (β-gal) from a lac operon. IPTG, a common allolactose mimic, is used as a chemical signal to induce transcription of genes from the lac promoter. The β-gal output is used to visualize gene expression patterns across printed surfaces when paired with substrates that produced insoluble dye when hydrolyzed. For instance, X-gal substrate is colorless when integrated into chemical signal solutions, yet produces localized areas of blue pigment where cleaved by β-gal. Similarly, the yellow substrate, Chlorophenol Red-β-D-galactopyranoside (CPRG), is cleaved to produce a red chromophore in areas of β-gal activity. These colorimetric stains allow for IPTG-induced gene expression to be assayed spatiotemporally across 3D objects.

In some use cases, this prototype facilitates fluorometric assays of gene expression as follows: Strains with fluorescent protein outputs are constructed. The strains IPTG/GFP, AHL/GFP, and RHA/GFP each express a green fluorescent protein (GFP) under the control of promoters LacI, LuxR, and RhaBAD when induced by the chemicals IPTG, N-acyl homoserine lactone (AHL), Rhamnose (RHA), respectively. For orthogonal AHL and IPTG patterning, the strain AHL/RFP is also cloned to produce a differentiable red fluorescent protein (RFP) under AHL-inducible control. Also, gene constructs using combinatorial promoters (where I̅P̅T̅G̅.̅A̅H̅L̅/GFP equated to NAND logic function and IPTG.AHL/GFP equated to AND logic function) were cloned for HLM objects performing spatial patterning from two-input cell logic. These transcriptional regulators may be utilized for multi-signal pattern generation.

In some use cases of this prototype, cells are applied onto or adjacent to printed structures as follows: A hydrogel composition of 1% agar/1% agarose/25% LB is heated to 100° C., and then cooled to 50° C. for the addition of antibiotic and cells. A bacterial suspension from overnight culture is normalized and added at 1:100 ratio to the hydrogel melt. The homogenized hydrogel-bacterial slurry is applied to the surface of printed objects. The hydrogel served several purposes including immobilizing the E. coli cells uniformly to 3D surfaces, providing a nutrient-rich, hydrated matrix for cellular tasks (e.g., proliferation, detection of extracellular signals, protein expression), and facilitating chemical signal diffusion from the printed object. The resulting object (including the polymer structure, hydrogel and living bacteria) is an HLM. The HLM incubates for 16 hours (37.5° C., 100% relative humidity (RH)).

In this prototype, resins enable multiple chemical signals to be independently controlled by print heads and directly embedded into printed objects. For instance, IPTG and AHL may each have signal orthogonality with bacterial strains of cognate signal-gated GFP reporters. Each chemical additive (IPTG and AHL) may be fully dissolved in a solvent (H₂O/DMSO) before integration into SUP705™ resin, to ensure that no solid particulate damages the print head. Solvent mixes of 1% v/v may be desirable, because they typically exhibit no significant changes (r.05) in shear properties (as compared to the original SUP705™ resin).

In a test of this prototype, different photopolymer resins (with embedded chemical signaling molecules) were created by mixing incremental concentrations and ratios of DMSO-solubilized inducer, IPTG, and uncured resin in low light. To rapidly manufacture test pieces, a VeroClear™ RGD810 tray was printed with 3×3 circular wells (10 mm diameter, 3 mm depth). Resin-IPTG mixtures generated for characterization tests were then pipetted into tray wells (180 μL), cured under UV light for 2 hours, and removed from tray with a scalpel. Testing of preservation of chemical signal functionality in cured photopolymer involved generating material test piece compositions with IPTG concentration (100 mM, 1M, 2.5M) and ratio to resin (10%, 5%, 1% volume) variants. LB-Amp plates were prepared for each test piece by applying 100 L IPTG (50 mM) in DMSO and 100 μL of a IPTG/GFP overnight stock in a 1:10 LB dilution. In this test, each test piece was placed in the center of an LB-Amp plate, incubated at 37° C. for 30 hours, and imaged on a blue light transilluminator.

In this prototype, biological functionality is preserved after a chemical signal had been incorporated into resin and exposed to UV-curing. For instance, in a test of this prototype, cured IPTG (2.0 M) and AHL (20 μM) resins generated a robust induction in respective E. coli strains (IPTG/GFP, AHL/GFP). In this prototype, IPTG (2.0 M, 0.1% v/v) and AHL (20 0.1% v/v) resins may be employed for direct printing.

In this prototype: (a) digital material descriptions in a multi-material printer guide bacterial gene expression across the surface of 3D printed objects; and (b) printed material gradients control release of chemical signals and facilitate the creation of complex, free-standing, 3D, multi-material, multifunctional hybrid living objects.

In this prototype: (a) augmented resins enable the 3D printer to pattern multiple chemical signal channels; (b) these inputs (chemical signals) regulate biologically computed outputs, and (c) the platform spatially patterns gene expression.

In this prototype, a computational model simulates chemical diffusion dynamics and biological response given a volumetric material distribution, thereby providing a predictive design tool for HLM fabrication.

An initial group of experiments was performed with this prototype, to test gene expression that is regulated by diffusible chemical signals which diffuse from a printed polymer object. To establish a spatial relationship between material-mediated chemical signal release and cellular response, a set of multi-material test templates were employed: Vero™ disks (50 mm-diameter, 3 mm height) with 10 mm “active” center regions of incremental material ratios (SUP705™:Vero™, 0-1.0, with 0.1 steps). The disks were prepared in an IPTG/X-Gal bath and incubated with a pUC19 E. coli hydrogel layer. Then β-gal gene expression (blue) was observed across the objects' surfaces.

In this initial group of experiments, Lad regulated expression correlated positively to the ratio of SUP705™ within the composite material, indicating that bacteria were responding proportionally to chemicals stored and released from those regions of the printed structure. Additionally, expression patterns characteristically extended outward from the active regions defined by the printer due to the diffusion of chemical signal, dispersing from the printed structure into the hydrogel surface layer. At higher SUP-ratios (>0.7), the pattern resembled a “halo” around the active region, with the color intensity maxima offset from the center of the template (discussed in modeling). As a negative control, when the active region was composed of Vero™ build material (SUP-ratio=0.0), negligible β-gal activity is observed. The spatial response of hybrid living objects (which comprised living cells and multi-material printed components) was repeatable and tunable to the SUP705™ material ratios defined by the print description.

In other tests of this prototype, CPRG was used in place of X-gal for another set of HLM test disks to examine spatiotemporal mechanisms. Time-lapse image capture of the incubation of HLMs (n=4) showed a period of CPRG diffusion from the active region (yellow, 0-18 hr). At hour 18, the β-gal-catalyzed colorimetric conversion of CPRG (magenta) first became visible to the eye, and continued to intensify and propagate (18-35 hr). These results indicated that the spatial and temporal behavior of the HLM templates relied on both the diffusion-mediated transport of chemical signal and the induction profile of engineered cells.

In this prototype, the digital material descriptions may also be used to produce other graded physical properties (in addition to chemical signal gradients) within a single object, including stiffness, solvent compatibility, and opacity. For instance, the 3D printer may create a solid polymer object with patterned and graded control of optical properties (i.e., transparency) and bioactive response (i.e., colorimetric β-gal gene expression indicators).

In a test, the prototype fabricated a “conformable” living device/bandage print using distributions of rigid (RGD810), rubber-like (FLX930), and chemical signals to exhibit site-specific/spatial control of bioactivity and flexibility. The resulting construct produced a specified bacterial response pattern across a variable flexible-to-rigid substrate. The substrate was configured to twist along one axis in such a way as provide ergonomic support or conformation to the body (e.g., for biomedical splints, sockets, or bedrests).

In another test, the prototype produced a printed object with a programmed, autonomous shape-change architecture. In this test, composites with varying solvent compatibilities (i.e. IPA-absorptivity) were distributed along longitudinal periodic gradients, in conjunction with rigid (RGD810) and chemical signaling materials, to produce both self-actuation and a patterned biosynthesized output.

In some use cases, this prototype deploys site-specific bacterial expression deterministically across complex 3D objects. This deterministic gene expression may be employed in bio-templated devices with mechanical, ergonomic, or dynamic functionality.

In this prototype: (a) the 3D printer may pattern multiple chemical signals that direct biologically computed outputs; (b) chemicals may be incorporated into a UV-curable photopolymer resin to create multiple chemical signaling materials; and (c) these signal channels may regulate gene expression systems to produce biologically driven spatial patterning that exhibits computational logic.

In this prototype, chemical signals may be directly deposited at the native resolution of the printer and multiple chemical signal channels may be independently controlled. For instance, IPTG and AHL chemical signaling resins may be compatible with printing and biological control. These resins may be loaded into the printer cartridges for fabrication of 3D objects with embedded chemical signal channels. Once printed, bacteria with specific engineered constructs may be introduced via the hydrogel layer.

In a test of this prototype, the 3D printer fabricated a bilobe-shaped rigid structure containing internal gradients of IPTG- and AHL-signaling material, and then a hydrogel layer containing living bacterial cells was applied to an external surface of the rigid structure. The living bacterial cells comprised a mixed population of cells containing genetic circuits IPTG/GFP and AHL/RFP. The hybrid structure (comprising the rigid structure and the hydrogel with bacterial cells) displayed a multi-output spatial response with green and red fluorescence, as a result of the templated IPTG and AHL chemical diffusion gradients. The expression of GFP and RFP was orthogonal.

In another test of this prototype, two additional bacterial response patterns were achieved by using two-input bioengineered logic. Specifically, gene circuits performing AND and NAND functions for IPTG- and AHL-inputs were responsive to the intersection of the two chemical channels. Induced spatial output was measured as a fluorescent intensity profile extending from the active region, with the AND instance showing a peak in midsection output, and the NAND instance showing an expected negation of this pattern.

In this prototype, a computational model predicts the biological response of HLMs on the basis of material distributions. The computational model takes into account: (a) diffusion of embedded chemical signals; (b) cellular response; and (c) 3D geometry of the HLM. (Recall that the 3D printer may create a solid polymer structure that has a complex 3D geometry).

The computational model employed in this prototype may be summarized as follows: Given a print description with volumetric material distribution data, the simulation first estimates HLM facilitated chemical signal diffusion. Initial inducer concentration is assigned based on measured volume and weight gain (for the swelling method) or the chemical signaling resin composition used (for the printing method). The transportation of a chemical signal over a regular closed surface Γ⊂ [symbol] ³ is approximated by

∂ₜc(x,t)=D∇_{Γ}²c(x,t)∀x∈Γ and t>0, (Equation 1)

c(x,0)=c₀(x)∀x∈Γ (Equation 2)

where ∇_{Γ}² is the surface Laplace-Beltrami operator, c(⋅, t):Γ→ [symbol] is the concentration of a diffused chemical signal, t≥0 is time, D is the diffusion coefficient, and c₀:Γ→ [symbol] is the initial concentration of the chemical signal.

Each chemical signal is assigned a diffusion coefficient D. For instance, the diffusion coefficient for IPTG is estimated to be

$${D}_{IPTG} = 3 \times {10}^{- 10 \frac{{m}^{2}}{s}} .$$

In this computation model (which is employed in the prototype), initial values for inducer are either known or are estimated from weight and area expansion of the hygroscopic swelling process. There are no boundary conditions in this model as it is assumed that the surface F is closed otherwise the represented shape would not be 3D printable.

In this computation model (which is employed in the prototype): (a) for spatial discretization, the regular surface is assigned a simplicial mesh; and (b) chemical signal concentration is approximated as a piecewise linear function upon this mesh. Specifically, for spatial discretization, assume that the regular surface F is a simplicial mesh M with vertices V_{M}={v₁ . . . , vₙ}, edges E_{M}={e₁, . . . , eₖ}, eⱼ∈V_{M}×V_{M} and faces F_{M}={f₁, . . . f_{M}}, fₖ∈V_{M}×V_{M}×V_{M} and embedding pᵢ=p(vᵢ):V_{M}→ [symbol] ³. The concentration may be approximated by a piecewise linear function cᵢ=c(vᵢ): V_{M}→ [symbol] on the mesh. The discrete Laplace-Beltrami operator of the concentration then is given as:

$${∇}^{2} c ( {v}_{i} ) = \frac{1}{2 {A}_{i}} {\sum }_{{v}_{j} ∈ N ( {v}_{i} )} ( cot {α}_{i , j} + cot {β}_{i , j} ) ( {c}_{j} - {c}_{i} ) \qquad ( Equation 3 )$$

where Aᵢ is one third the area of all triangles incident on vᵢ, N(vᵢ) are the neighboring vertices connected by and edge, and α_{i,j} and β_{i,j} are the angles opposing the corresponding edge.

Since the Laplacian is a linear operator, this can be written as Lc, where L∈ [symbol] ^{|V|×|V|} is the matrix of cotangent coefficients and c∈ [symbol] ^{|V|} is the piecewise linear approximation of concentrations over the surface. For temporal discretization, we aim for large time steps essential for fast design iteration. Thus, for temporal discretization of (1) we can facilitate an implicit integration scheme such that c(t+h)=c(t)+hDc(t+h)⇔(I−hDL)c(t+h)=c(t).

In this computation model (which is employed in the prototype), diffused chemical concentrations are mapped to GFP response through response curves. For instance, in a test of the prototype, response curves were obtained by measuring relative GFP fluorescence response of IPTG/GFP and AHL/GFP reporter strains for predefined inducer concentrations. Fluorescence measurements were collected by a 96-well fluorescent plate reader (Ex 485 nm, Em 528 nm).

In some tests of this prototype: (a) chemical signaling resin samples were prepared by mechanically mixing 2M IPTG-DMSO solution into pure liquid (uncured) SUP705™ resin; and (b) 10 mL of resin sample were loaded for each test.

The prototype (and test results for the prototype) described in the preceding 38 paragraphs are non-limiting examples of this invention. This invention may be implemented in many different ways.

### Examples of Use Cases

In some use cases of the present invention, the 3D-printed structure controls complex systems. In these complex systems, multiple different bioactive materials may be released from the 3D-printed structure and may affect gene expression in the engineered organisms. Here are five, non-limiting examples:

### First Example—Salmonella Detection

The present invention may function as a Salmonella-reporting countertop or packaging material. The countertop or food packaging may be printed from a 3DP material with a pattern of chemical inducer arabinose, localized to “problem areas”, such as the perimeter of a sink or the inner lining of an egg carton. An engineered bacteriophage ΦSH19, specific to virulent Salmonella strains, may be embedded as the biological construct. In the presence of Salmonella, the integrated phage may act as a vector to insert an engineered gene construct encoding a reporter gene (i.e., lycopene, a red pigment) under the control of arabinose, to the Salmonella cell. Thus, Salmonella in contact with ‘problem areas’ of the egg carton or counter top may express the gene construct, localized to the chemical inducer, and produce a colorimetric indicator—alerting the user of the presence of Salmonella.

### Second Example—Fruit Ripening

A 3D print in the shape of an aerated fruit crate, or insert, may have regions around each fruit graded with a chemical inducer, vanillic acid. Using Saccharomyces cerevisiae yeast, engineered to produce Ethylene gas controlled by vanillic acid, food distributors may hasten the ripening of fruit just before it reaches consumer end points, such as the grocery store. The spatial gradation of vanillic acid may ensure that fruit ripens more in the mid-region, and less on its ends where it is likely to get bruised, while engineered negative-feedback of the vanillic acid repressor gene, VanR, may ensure that Ethylene gas is always expressed below a certain range.

### Third Example—Pigment

Two populations of engineered cells may be grown on the 3D printed material, for instance two kinds of E. coli. E. coli type A nay be induced by the chemical IPTG and produce tryptophan. E. coli type B may be induced by tryptophan and produce a pigment. The 3D printed material may release IPTG. Having two engineered cell populations may facilitate more complex interactions in the production of the final pigment, and may enable modulation of the strength of the signal in different ways (e.g. a small amount of IPTG may produce a large amount of tryptophan which then makes a large amount of pigment).

### Fourth Example—Melanin

Two populations of engineered cells may be grown on the 3D printed material. For instance, the two populations may comprise two types of E. coli. E, where: (a) the first type is induced by IPTG and creates tyrosine; and (b) the second type is induced by tyrosine and, in the presence of copper sulfate, produces melanin. The 3D printed material may release copper sulfate and IPTG.

### Fifth Example—Repressor

In some cases, instead of induction, a first cell type produces a repressor, which reduces activity and biosynthesis by a second cell type. Cells may also be used with quorum sensing mechanisms that involve autoinducers. The autoinducers may repress or activate genes when they reach a threshold.

### Computers

In illustrative implementations of this invention, one or more computers (e.g., servers, network hosts, client computers, integrated circuits, microcontrollers, controllers, field-programmable-gate arrays, personal computers, digital computers, driver circuits, or analog computers) are programmed or specially adapted to perform one or more of the following tasks: (1) to control the operation of, or interface with, hardware components of a 3D printer, including any actuator, printhead, or pump; (2) to control the mixing or blending multiple materials to produce mixtures or blends that may deposited by a 3D printer; (3) to create a digital model of a solid polymer structure; (4) to generate print instructions (e.g., droplet deposition instructions) based on the digital model; (5) to receive data from, control, or interface with one or more sensors; (6) to perform any other calculation, computation, program, algorithm, or computer function described or implied herein; (7) to receive signals indicative of human input; (8) to output signals for controlling transducers for outputting information in human perceivable format; (9) to process data, to perform computations, and to execute any algorithm or software; and (10) to control the read or write of data to and from memory devices (tasks 1-10 of this sentence referred to herein as the “Computer Tasks”). The one or more computers (e.g. 241) may, in some cases, communicate with each other or with other devices: (a) wirelessly, (b) by wired connection, (c) by fiber-optic link, or (d) by a combination of wired, wireless or fiber optic links.

In exemplary implementations, one or more computers are programmed to perform any and all calculations, computations, programs, algorithms, computer functions and computer tasks described or implied herein. For example, in some cases: (a) a machine-accessible medium has instructions encoded thereon that specify steps in a software program; and (b) the computer accesses the instructions encoded on the machine-accessible medium, in order to determine steps to execute in the program. In exemplary implementations, the machine-accessible medium may comprise a tangible non-transitory medium. In some cases, the machine-accessible medium comprises (a) a memory unit or (b) an auxiliary memory storage device. For example, in some cases, a control unit in a computer fetches the instructions from memory.

In illustrative implementations, one or more computers execute programs according to instructions encoded in one or more tangible, non-transitory, computer-readable media. For example, in some cases, these instructions comprise instructions for a computer to perform any calculation, computation, program, algorithm, or computer function described or implied herein. For example, in some cases, instructions encoded in a tangible, non-transitory, computer-accessible medium comprise instructions for a computer to perform the Computer Tasks.

### Network Communication

In illustrative implementations of this invention, electronic devices (e.g., 241) are each configured for wireless or wired communication with other devices in a network.

For example, in some cases, one or more of these electronic devices each include a wireless module for wireless communication with other devices in a network. Each wireless module may include (a) one or more antennas, (b) one or more wireless transceivers, transmitters or receivers, and (c) signal processing circuitry. Each wireless module may receive and transmit data in accordance with one or more wireless standards.

In some cases, one or more of the following hardware components are used for network communication: a computer bus, a computer port, network connection, network interface device, host adapter, wireless module, wireless card, signal processor, modem, router, cables or wiring.

In some cases, one or more computers (e.g., 241) are programmed for communication over a network. For example, in some cases, one or more computers are programmed for network communication: (a) in accordance with the Internet Protocol Suite, or (b) in accordance with any other industry standard for communication, including any USB standard, ethernet standard (e.g., IEEE 802.3), token ring standard (e.g., IEEE 802.5), or wireless communication standard, including IEEE 802.11 (Wi-Fi®), IEEE 802.15 (Bluetooth®/Zigbee®), IEEE 802.16, IEEE 802.20, GSM (global system for mobile communications), UMTS (universal mobile telecommunication system), CDMA (code division multiple access, including IS-95, IS-2000, and WCDMA), LTE (long term evolution), or 5G (e.g., ITU IMT-2020).

### Actuators

In illustrative implementations, the 3D printer includes actuators (e.g., 220). Each actuator (including each actuator for actuating any movement) may be any kind of actuator, including a linear, rotary, electrical, piezoelectric, electro-active polymer, mechanical or electro-mechanical actuator. In some cases, the actuator includes and is powered by an electrical motor, including any stepper motor or servomotor. In some cases, the actuator includes a gear assembly, drive train, pivot, joint, rod, arm, or other component for transmitting motion. In some cases, one or more sensors are used to detect position, displacement or other data for feedback to one of more of the actuators.

### Definitions

The terms “a” and “an”, when modifying a noun, do not imply that only one of the noun exists. For example, a statement that “an apple is hanging from a branch”: (i) does not imply that only one apple is hanging from the branch; (ii) is true if one apple is hanging from the branch; and (iii) is true if multiple apples are hanging from the branch.

To compute “based on” specified data means to perform a computation that takes the specified data as an input.

The term “comprise” (and grammatical variations thereof) shall be construed as if followed by “without limitation”. If A comprises B, then A includes B and may include other things.

A digital computer is a non-limiting example of a “computer”. An analog computer is a non-limiting example of a “computer”. A computer that performs both analog and digital computations is a non-limiting example of a “computer”. However, a human is not a “computer”, as that term is used herein.

“Computer Tasks” is defined above.

“Defined Term” means a term or phrase that is set forth in quotation marks in this Definitions section.

“DNA” means deoxyribonucleic acid.

For an event to occur “during” a time period, it is not necessary that the event occur throughout the entire time period. For example, an event that occurs during only a portion of a given time period occurs “during” the given time period.

The term “e.g.” means for example.

The fact that an “example” or multiple examples of something are given does not imply that they are the only instances of that thing. An example (or a group of examples) is merely a non-exhaustive and non-limiting illustration.

Unless the context clearly indicates otherwise: (1) a phrase that includes “a first” thing and “a second” thing does not imply an order of the two things (or that there are only two of the things); and (2) such a phrase is simply a way of identifying the two things, respectively, so that they each may be referred to later with specificity (e.g., by referring to “the first” thing and “the second” thing later). For example, unless the context clearly indicates otherwise, if an equation has a first term and a second term, then the equation may (or may not) have more than two terms, and the first term may occur before or after the second term in the equation. A phrase that includes a “third” thing, a “fourth” thing and so on shall be construed in like manner.

“For instance” means for example.

To say a “given” X is simply a way of identifying the X, such that the X may be referred to later with specificity. To say a “given” X does not create any implication regarding X. For example, to say a “given” X does not create any implication that X is a gift, assumption, or known fact.

“Herein” means in this document, including text, specification, claims, abstract, and drawings.

As used herein: (1) “implementation” means an implementation of this invention; (2) “embodiment” means an embodiment of this invention; (3) “case” means an implementation of this invention; and (4) “use scenario” means a use scenario of this invention.

The term “include” (and grammatical variations thereof) shall be construed as if followed by “without limitation”.

Unless the context clearly indicates otherwise, “or” means and/or. For example, A or B is true if A is true, or B is true, or both A and B are true. Also, for example, a calculation of A or B means a calculation of A, or a calculation of B, or a calculation of A and B.

A parenthesis is simply to make text easier to read, by indicating a grouping of words. A parenthesis does not mean that the parenthetical material is optional or may be ignored.

“Polymer structure” means a structure that comprises one or more polymers.

“RNA” means ribonucleic acid.

As used herein, the term “set” does not include a group with no elements.

Unless the context clearly indicates otherwise, “some” means one or more.

As used herein, a “subset” of a set consists of less than all of the elements of the set.

The term “such as” means for example.

To say that a machine-readable medium is “transitory” means that the medium is a transitory signal, such as an electromagnetic wave.

“UV” means ultraviolet.

Except to the extent that the context clearly requires otherwise, if steps in a method are described herein, then the method includes variations in which: (1) steps in the method occur in any order or sequence, including any order or sequence different than that described herein; (2) any step or steps in the method occur more than once; (3) any two steps occur the same number of times or a different number of times during the method; (4) any combination of steps in the method is done in parallel or serially; (5) any step in the method is performed iteratively; (6) a given step in the method is applied to the same thing each time that the given step occurs or is applied to different things each time that the given step occurs; (7) one or more steps occur simultaneously; or (8) the method includes other steps, in addition to the steps described herein.

Headings are included herein merely to facilitate a reader's navigation of this document. A heading for a section does not affect the meaning or scope of that section.

This Definitions section shall, in all cases, control over and override any other definition of the Defined Terms. The Applicant or Applicants are acting as his, her, its or their own lexicographer with respect to the Defined Terms. For example, the definitions of Defined Terms set forth in this Definitions section override common usage and any external dictionary. If a given term is explicitly or implicitly defined in this document, then that definition shall be controlling, and shall override any definition of the given term arising from any source (e.g., a dictionary or common usage) that is external to this document. If this document provides clarification regarding the meaning of a particular term, then that clarification shall, to the extent applicable, override any definition of the given term arising from any source (e.g., a dictionary or common usage) that is external to this document. Unless the context clearly indicates otherwise, any definition or clarification herein of a term or phrase applies to any grammatical variation of the term or phrase, taking into account the difference in grammatical form. For example, the grammatical variations include noun, verb, participle, adjective, and possessive forms, and different declensions, and different tenses.

### Variations

This invention may be implemented in many different ways. Here are some non-limiting examples:

In some implementations, this invention is a method comprising: (a) fabricating a solid polymer structure in such a way that (i) diffusible chemical signals are embedded in the solid polymer structure during the fabricating, (ii) concentration of each of the chemical signals at each of a set of spatial regions of the polymer structure is controlled during the fabricating and varies as a function of spatial position in the polymer structure, and (iii) each of the chemical signals is deposited in a specific spatial pattern of concentrations in the solid polymer structure, which specific spatial pattern is different than that of at least some of the other chemical signals; (b) causing a hydrogel to be in physical contact with a surface of the solid polymer structure, which hydrogel surrounds non-human living organisms that contain recombinant DNA; and (c) after a portion of the diffusible chemical signals diffuse out of the solid polymer structure and into the hydrogel, regulating, with the chemical signals, gene expression by the living organisms. In some cases, a region of the solid polymer structure has a hardness, on Shore Durometer Scale D, that is greater than or equal to 80. In some cases: (a) a first region of the solid polymer structure has a hardness, on Shore Durometer Scale D, that is greater than or equal to 80; and (b) a second region of the solid polymer structure has a hardness, on Shore Durometer Scale A, that is greater than or equal to 20 and less than or equal to 77. In some cases, a region of an external surface of the solid polymer structure: (a) is at least one square millimeter in area; and (b) has a surface roughness that is greater than or equal to 0.8 microns R_{α} and less than or equal to 1.2 microns R_{α}. In some cases: (a) at least one region of the solid polymer structure is a rectangular cuboid and has a volume that is greater than or equal to 1 microliter; (b) the average diameter of cavities, if any, in the region is less than 1 micron; and (c) the average diameter of pores, if any, in the region is less than 1 micron. In some cases: (a) at least one region of the solid polymer structure is a rectangular cuboid and has a volume that is greater than or equal to 1 microliter; (b) the average diameter of cavities, if any, in the region is less than 100 nanometers; and (c) the average diameter of pores, if any, in the region is less than 100 nanometers. In some cases, the living organisms are single-celled. In some cases, the diffusible chemical signals control gene expression of the recombinant DNA. In some cases, the solid polymer structure includes acrylate compounds. In some cases, the living organisms do not enter into the solid polymer structure. Each of the cases described above in this paragraph is an example of the method described in the first sentence of this paragraph, and is also an example of an embodiment of this invention that may be combined with other embodiments of this invention.

In some implementations, this invention is a system comprising: (a) a solid polymer structure; (b) diffusible chemical signals; (c) a hydrogel; and (d) non-human living organisms that contain recombinant DNA; wherein (i) the diffusible chemical signals are embedded in the solid polymer structure, (ii) concentration of each of the chemical signals at each of a set of spatial regions of the polymer structure varies as a function of spatial position in the polymer structure, (iii) each of the chemical signals is present in a specific spatial pattern of concentrations in the solid polymer structure, which specific spatial pattern is different than that of at least some of the other chemical signals, (iv) the hydrogel is in physical contact with a surface of the solid polymer structure, and (v) the hydrogel surrounds the living organisms. In some cases, the system is configured in such a way that, after a portion of the diffusible chemical signals diffuse out of the solid polymer structure and into the hydrogel, the chemical signals regulate gene expression by the living organisms. In some cases, the solid polymer structure includes acrylate compounds. In some cases, the chemical signals are configured to control gene expression by the recombinant DNA. In some cases, a region the solid polymer structure has a hardness, on Shore Durometer Scale D, that is greater than or equal to 80 and less than or equal to 88. In some cases, a region of an external surface of the solid polymer structure: (a) is at least one square millimeter in area; and (b) has a surface roughness that is greater than or equal to 0.8 microns R_{α} and less than or equal to 1.2 microns R_{α}. In some cases, a region of an external surface of the polymer structure: (a) is at least one square millimeter in area; and (b) does not have any feature that deviates in height by more than 1 micron from the mean height of the region. In some cases: (a) a region of the solid polymer structure is a rectangular cuboid and has a volume that is greater than or equal to 1 microliter; (b) the average diameter of cavities, if any, in the region is less than 1 micron; and (c) the average diameter of pores, if any, in the region is less than 1 micron. In some cases: (a) a region of the solid polymer structure is a rectangular cuboid and has a volume that is greater than or equal to 1 microliter; (b) the average diameter of cavities, if any, in the region is less than 100 nanometers; and (c) the average diameter of pores, if any, in the region is less than 100 nanometers. Each of the cases described above in this paragraph is an example of the system described in the first sentence of this paragraph, and is also an example of an embodiment of this invention that may be combined with other embodiments of this invention.

In some implementations, this invention is an apparatus comprising a computer-controlled three-dimensional (3D) printer, which 3D printer is configured to perform fabrication of a solid polymer structure, in such a way that: (a) during the fabrication, the 3D printer deposits polymers that include acrylate compounds; (b) during the fabrication, the 3D printer deposits diffusible chemical signals, which chemical signals are compounds that are inducers for gene expression in specific living organisms; (c) as a result of the fabrication (i) concentration of each of the chemical signals at each of a set of spatial regions of the polymer structure varies as a function of spatial position in the polymer structure, and (ii) each of the chemical signals is present in a specific spatial pattern of concentrations in the solid polymer structure, which specific spatial pattern is different than that of at least some of the other chemical signals; and (d) after the polymers cure, a region of the solid polymer structure has a hardness, on Shore Durometer Scale D, that is greater than or equal to 80 and less than or equal to 88. The apparatus described in the first sentence of this paragraph may be combined with other embodiments of this invention.

Each description herein (or in the Provisional) of any method, apparatus or system of this invention describes a non-limiting example of this invention. This invention is not limited to those examples, and may be implemented in other ways.

Each description herein (or in the Provisional) of any prototype of this invention describes a non-limiting example of this invention. This invention is not limited to those examples, and may be implemented in other ways.

Each description herein (or in the Provisional) of any implementation, embodiment or case of this invention (or any use scenario for this invention) describes a non-limiting example of this invention. This invention is not limited to those examples, and may be implemented in other ways.

Each Figure, diagram, schematic or drawing herein (or in the Provisional) that illustrates any feature of this invention shows a non-limiting example of this invention. This invention is not limited to those examples, and may be implemented in other ways.

The above description (including without limitation any attached drawings and figures) describes illustrative implementations of the invention. However, the invention may be implemented in other ways. The methods and apparatus which are described herein are merely illustrative applications of the principles of the invention. Other arrangements, methods, modifications, and substitutions by one of ordinary skill in the art are also within the scope of the present invention. Numerous modifications may be made by those skilled in the art without departing from the scope of the invention. Also, this invention includes without limitation each combination and permutation of one or more of the items (including hardware, hardware components, methods, processes, steps, software, algorithms, features, or technology) that are described herein.

What is claimed:

1. A method comprising:
(a) fabricating a solid polymer structure in such a way that (i) diffusible chemical signals are embedded in the solid polymer structure during the fabricating, (ii) concentration of each of the chemical signals at each of a set of spatial regions of the polymer structure is controlled during the fabricating and varies as a function of spatial position in the polymer structure, and (iii) each of the chemical signals is deposited in a specific spatial pattern of concentrations in the solid polymer structure, which specific spatial pattern is different than that of at least some of the other chemical signals;
(b) causing a hydrogel to be in physical contact with a surface of the solid polymer structure, which hydrogel surrounds non-human living organisms that contain recombinant DNA; and
(c) after a portion of the diffusible chemical signals diffuse out of the solid polymer structure and into the hydrogel, regulating, with the chemical signals, gene expression by the living organisms.

2. The method of claim 1, wherein a region of the solid polymer structure has a hardness, on Shore Durometer Scale D, that is greater than or equal to 80.

3. The method of claim 1, wherein:
(a) a first region of the solid polymer structure has a hardness, on Shore Durometer Scale D, that is greater than or equal to 80; and
(b) a second region of the solid polymer structure has a hardness, on Shore Durometer Scale A, that is greater than or equal to 20 and less than or equal to 77.

4. The method of claim 1, wherein a region of an external surface of the solid polymer structure:
(a) is at least one square millimeter in area; and
(b) has a surface roughness that is greater than or equal to 0.8 microns R_{α} and less than or equal to 1.2 microns R_{α}.

5. The method of claim 1, wherein:
(a) at least one region of the solid polymer structure is a rectangular cuboid and has a volume that is greater than or equal to 1 microliter;
(b) the average diameter of cavities, if any, in the region is less than 1 micron; and
(c) the average diameter of pores, if any, in the region is less than 1 micron.

6. The method of claim 1, wherein:
(a) at least one region of the solid polymer structure is a rectangular cuboid and has a volume that is greater than or equal to 1 microliter;
(b) the average diameter of cavities, if any, in the region is less than 100 nanometers; and
(c) the average diameter of pores, if any, in the region is less than 100 nanometers.

7. The method of claim 1, wherein the living organisms are single-celled.

8. The method of claim 1, wherein the diffusible chemical signals control gene expression of the recombinant DNA.

9. The method of claim 1, wherein the solid polymer structure includes acrylate compounds.

10. The method of claim 1, wherein the living organisms do not enter into the solid polymer structure.

11. A system comprising:
(a) a solid polymer structure;
(b) diffusible chemical signals;
(c) a hydrogel; and
(d) non-human living organisms that contain recombinant DNA;
wherein
(i) the diffusible chemical signals are embedded in the solid polymer structure,
(ii) concentration of each of the chemical signals at each of a set of spatial regions of the polymer structure varies as a function of spatial position in the polymer structure,
(iii) each of the chemical signals is present in a specific spatial pattern of concentrations in the solid polymer structure, which specific spatial pattern is different than that of at least some of the other chemical signals,
(iv) the hydrogel is in physical contact with a surface of the solid polymer structure, and
(v) the hydrogel surrounds the living organisms.

12. The system of claim 11, wherein the system is configured in such a way that, after a portion of the diffusible chemical signals diffuse out of the solid polymer structure and into the hydrogel, the chemical signals regulate gene expression by the living organisms.

13. The system of claim 11, wherein the solid polymer structure includes acrylate compounds.

14. The system of claim 11, wherein the chemical signals are configured to control gene expression by the recombinant DNA.

15. The system of claim 11, wherein a region the solid polymer structure has a hardness, on Shore Durometer Scale D, that is greater than or equal to 80 and less than or equal to 88.

16. The system of claim 11, wherein a region of an external surface of the solid polymer structure:
(a) is at least one square millimeter in area; and
(b) has a surface roughness that is greater than or equal to 0.8 microns R_{α} and less than or equal to 1.2 microns R_{α}.

17. The system of claim 11, wherein a region of an external surface of the polymer structure:
(a) is at least one square millimeter in area; and
(b) does not have any feature that deviates in height by more than 1 micron from the mean height of the region.

18. The system of claim 11, wherein:
(a) a region of the solid polymer structure is a rectangular cuboid and has a volume that is greater than or equal to 1 microliter;
(b) the average diameter of cavities, if any, in the region is less than 1 micron; and
(c) the average diameter of pores, if any, in the region is less than 1 micron.

19. The system of claim 11, wherein:
(a) a region of the solid polymer structure is a rectangular cuboid and has a volume that is greater than or equal to 1 microliter;
(b) the average diameter of cavities, if any, in the region is less than 100 nanometers; and
(c) the average diameter of pores, if any, in the region is less than 100 nanometers.

20. An apparatus comprising a computer-controlled three-dimensional (3D) printer, which 3D printer is configured to perform fabrication of a solid polymer structure, in such a way that:
(a) during the fabrication, the 3D printer deposits polymers that include acrylate compounds;
(b) during the fabrication, the 3D printer deposits diffusible chemical signals, which chemical signals are compounds that are inducers for gene expression in specific living organisms;
(c) as a result of the fabrication
(i) concentration of each of the chemical signals at each of a set of spatial regions of the polymer structure varies as a function of spatial position in the polymer structure, and
(ii) each of the chemical signals is present in a specific spatial pattern of concentrations in the solid polymer structure, which specific spatial pattern is different than that of at least some of the other chemical signals; and
(d) after the polymers cure, a region of the solid polymer structure has a hardness, on Shore Durometer Scale D, that is greater than or equal to 80 and less than or equal to 88.
